(19) United States
(12) Patent Application Publication
(10) Pub. No.: US 2006/0281791 A1
(43) Pub. Date: Dec. 14, 2006

(54) Methods of increasing proliferation of adult mammalian cardiomyocytes through p38 map kinase inhibition

(75) Inventors: Mark T. Keating, Weston, MA (US); Felix B. Engel, Boston, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: CHILDREN'S MEDICAL CENTER CORPORATION, Boston, MA (US)

(21) Appl. No.: 11/414,733

(22) Filed: Apr. 28, 2006

Related U.S. Application Data
(60) Provisional application No. 60/676,117, filed on Apr. 29, 2005.

(51) Int. Cl. A61K 31/4412

(52) U.S. Cl. 514/355

(57) ABSTRACT

Compositions and methods for increasing proliferation and/or de-differentiation of postmitotic mammalian cardiomyocytes are disclosed to slow, reduce, or prevent the onset of cardiac damage. In addition, the methods and compositions of the invention can used to produce de-differentiated cardiomyocytes, which can then be used in tissue grafting, implantation or transplantation. The invention is based, in part, on the discovery that postmitotic mammalian cardiomyocytes can proliferate as a result of targeted disruption of p38 MAP kinase. p38 inhibition with optional growth factor stimulation can induce cytokinesis in adult cardiomyocytes.

### REFERENCE TO RELATED APPLICATION

The present application claims priority to U.S. Provisional Patent Application Ser. No. 60/676,117 entitled “Methods Of Increasing Proliferation Of Adult Mammalian Cardiomyocytes Through P38 Map Kinase Inhibition,” filed on Apr. 29, 2005, which is herein incorporated by reference.

### BACKGROUND OF THE INVENTION

Highly differentiated mammalian cells are thought to be incapable of proliferation. These cells have exited the cell cycle. Proteins critical for cellular specialisation have accumulated and driven these cells to their final form and function (Studzinski and Harrison 1999 Int Rev Cytol 189: 1-58). In contrast with mammals, differentiated cells in teleost fish (Poss et al. 2003 Dev Dyn 226: 202-10) and urodele amphibians (Brockes and Kumar 2002 Nat Rev Mol Cell Biol 3: 566-74) can dedifferentiate and/or proliferate, enabling regeneration. For example zebrafish hearts regenerate through cardiomyocyte proliferation (Poss et al. 2002 Science 298: 2188-90). Thus, a thorough understanding of mechanisms regulating cell cycle exit, and the development of approaches to reactivate proliferation of mammalian cells, would be of great therapeutic value.

Mammalian cardiac regeneration has been studied since the mid-nineteenth century. The consistent conclusion of these studies has been that the heart has little or no regenerative capacity (Rumyantsev 1977 Int Rev Cytol 51: 186-273; Mummery 2005 Nature 433: 585-7). This is a major medical problem, as ischaemic heart disease, resulting in cardiac muscle loss, is the leading cause of morbidity and mortality among adults aged 60 and older, and the second most common cause of death in ages 15 to 59. Approximately 17 million people die of cardiovascular disease every year according to the World Health Report 2003.

Accordingly, there is a need in the art for methods of increasing and/or promoting proliferation of adult mammalian cardiomyocytes.

### SUMMARY OF THE INVENTION

The present invention provides compositions and methods for increasing proliferation and/or de-differentiation of postmitotic mammalian cardiomyocytes. The invention can be used to slow, reduce, or prevent the onset of cardiac damage caused by, for example, myocardial ischemia, hypoxia, stroke, or myocardial infarction. In addition, the methods and compositions of the invention can used to produce de-differentiated cardiomyocytes, which can then be used in tissue grafting.

The invention is based, in part, on the discovery that postmitotic mammalian cardiomyocytes can proliferate. One mechanism of cell cycle regulation for mammalian cardiomyocytes is p38 activity; that is p38 is a key negative regulator of mammalian cardiomyocyte division. p38 activity is inversely correlated with cardiac growth during development, and its overexpression blocks proliferation of fetal cardiomyocytes in vitro. Genetic activation of p38 in vivo reduces fetal cardiomyocytes proliferation, whereas targeted disruption of p38w increases neonatal cardiomyocyte mitoses. Growth factor stimulation and p38 inhibition can induce cytokinesis in adult cardiomyocytes. Growth factors useful in conjunction with p38 inhibitors in clued FGF1, IL-1β, and NRG-1-β1 as well as factors listed in Table S-2. These results indicate that the inhibitory effects of p38 on cardiomyocyte proliferation are reversible and that postmitotic, differentiated cells are capable of proliferation.

### BRIEF DESCRIPTION OF THE DRAWINGS

FIG. 1 is a graph of cardiac growth and p38 activity versus developmental time. The rate of cardiac growth (black line) was inversely correlated with p38 activity (bars, n=5, mean ±SD). p38 activity was measured by its ability to phosphorylate ATF-2. p38 activity was biphasic during development, low at E12 and E19, and high at E15 and E21-adult.

FIGS. 2A-2C are graphs demonstrating that p38α regulates neonatal cardiomyocyte proliferation potential. Neonatal rat cardiomyocytes were stimulated with FGF1, IL-1β, and/or NRG-1-β1 with or without p38 inhibition, and analyzed for DNA synthesis (BrdU) or karyokinesis (H3P). FIG. 2A shows p38i increased growth factor-induced DNA synthesis. Note that 80.4±4.4% of cardiomyocytes were BrdU-positive after stimulation with FGF1, NRG-1-β1 (NRG) and 10 μM p38i (n=3, mean±SD, p<0.01). Diluent for p38i was DMSO. FIG. 2B shows that dominant negativeinhibition of p38α, but not p38β, increased FGF1-induced BrdU incorporation (p<0.01, DN=adenoviral infection with dominant negative constructs, low=100 PFU/cell, high=500 PFU/cell). Diluent for FGF1 was 0.1% BSA/PBS. FIG. 2C shows that p38 inhibition significantly increased growth factor-induced karyokinesis (n=3, mean±SD, p<0.01).

FIG. 3 is a graph demonstrating that p38 controls neonatal cardiomyocyte proliferation. Neonatal cardiomyocyte proliferation was analyzed by cell count, FACS, BrdU, H3P, survivin and aurora B staining. In FIG. 3, p38 inhibition augmented growth factor-induced cardiomyocyte proliferation as measured by cell count (n=2 or 3 for each time point, mean±SD, day 3: p<0.05, day 4: and 5: p<0.01). Note that a single stimulation with FGF1 and IL-1β in the presence of p38i increased cardiomyocyte numbers by 2.6-fold after 5 days of stimulation.

FIGS. 4A-4C demonstrate that adult cardiomyocyte proliferation is controlled by p38. Adult rat cardiomyocytes were analyzed using BrdU, H3P and aurora B. In FIG. 4A, p38 inhibition increased growth factor-induced DNA synthesis (BrdU) in adult cardiomyocytes (n=3, mean±SD, p<0.01). In FIG. 4B, mitotic activity (H3P) in adult cardiomyocytes was increased by p38 inhibition (n=4, mean±SD, p<0.01). In FIG. 4C, adult cardiomyocytes undergo cytokinesis (aurora B) when incubated with growth factors and p38i (n=4, mean±SD, p<0.01).

FIGS. 5A-5C compare the effects of a variety of p38 inhibitors on adult rat cardiomyocytes using Ki67, BrdU, and H3P. FIG. 5A shows the percentage of Ki67-positive neonatal cardiomyocytes. FIG. 5B shows the percentage of BrdU-positive neonatal cardiomyocytes and FIG. 5c shows the percentage of H3P-positive neonatal cardiomyocytes.

FIG. 6 demonstrates the effect of a p38 inhibitor on fractional shorting (FS) as a measure of systolic function one day after myocardial infarct. The sham-operated animals showed no significant changes in FS. The control (MI) showed a decrease in FS after myocardial infarct. However, the decrease in FS was significantly reduced when p38 inhibitor was given after MI. Fractional shorting (FS) is calculated as a measure of systolic function, according to the M-mode tracing from the cross-sectional view: maximal LV end-diastolic diameter (at the time of maximal cavity dimension), minimal LV end-systolic diameter (at the time of maximum anterior motion of the posterior wall), FS (%)={(LVEDD-LVESD)/LVEDD}×100.

FIG. 7 is a graph demonstrating the effect of a p38 inhibitor on fractional shorting (FS) 14 days after myocardial infarct.

FIG. 8 is a graph demonstrating that combined administration of FGF1 and a p38 inhibitor induced cardiomyocyte mitosis in vivo.

FIGS. 9A-9D are graphs demonstrating that combined administration of FGF1 and a p38 inhibitor improves heart function. FIG. 9A is a graph of percentage fractional shortening at 1 day; FIG. 9B is a graph of percentage fractional shortening at 2 weeks; FIG. 9C is a graph of percentage scar volume and FIG. 9D is a graph of the thining index for various treatments.

FIGS. 10A-10E are graphs demonstrating that combined administration of FGF1 and a p38 inhibitor improves heart function permanently. FIG. 10A is a graph of percentage fractional shortening at 1 day; FIG. 10B is a graph of percentage fractional shortening at 3 months; FIG. 10C is a graph of percentage scar volume; FIG. 10D is a graph of the thining index for various treatments and FIG. 10E is a graph comparing percentage fractional shortening at 1 month and 3 months.

FIG. 11 is a graph demonstrating that combined administration of FGF1 and a p38 inhibitor increases vascularization.

FIGS. 12A-12E provide experimental data for animal sacrificed at 2 weeks. FIG. 12A is a graph illustrating percentage fractional shortening. FIG. 12B is a graph of scar volume. FIG. 12C shows percentage muscle loss. FIG. 12D shows thinning index measurements and FIG. 12E shows wall thickness.

FIGS. 13A-13E provide experimental data for animal sacrificed at 3 months. FIG. 13A is a graph illustrating percentage fractional shortening. FIG. 13B is a graph of scar volume. FIG. 13C shows percentage muscle loss. FIG. 13D shows thinning index measurements and FIG. 13E shows wall thickness.

### DETAILED DESCRIPTION OF THE INVENTION

In one aspect, the invention provides methods of inducing adult mammalian cardiomyocytes to divide. Adult mammalian cardiomyocytes are considered terminally differentiated and incapable of proliferation. Consequently, acutely injured mammalian hearts do not regenerate, they scar. One important mechanism used by mammalian cardiomyocytes to control cell cycle is p38 MAP kinase activity. p38 regulates expression of genes required for mitosis in cardiomyocytes, including cyclin A and cyclin B. p38 activity is inversely correlated with cardiac growth during development, and its overexpression blocks fetal cardiomyocyte proliferation. Activation of p38 in vivo by MKK3bE reduces BrdU incorporation in fetal cardiomyocytes by 17.6%. By contrast, cardiac-specific p38α knockout mice show a 92.3% increase in neonatal cardiomyocyte mitoses. Furthermore, inhibition of p38 in adult cardiomyocytes promotes cytokinesis. Mitosis in adult cardiomyocytes is associated with transient dedifferentiation of the contractile apparatus. The present invention demonstrates that p38 is a key negative regulator of cardiomyocyte proliferation and indicate that adult cardiomyocytes can divide.

In contrast to adult cardiomyocytes, mammalian cardiomyocytes do proliferate during fetal development. Shortly after birth, these cardiomyocytes downregulate cell cycle-perpetuating factors like cyclin A and cdk2. The loss of proliferation capacity coincides with increased levels of the cell cycle inhibitors p21 and p27. At this point of development, postnatal cardiac growth is mediated by cardiomyocyte hypertrophy. This transition from hyperplastic to hypertrophic growth is characterised by maturation of the contractile apparatus, a cytoplasmic structure that is thought to preclude cytokinesis (Rumyantsev 1977 Int Rev Cytol 51: 186-273). Thus, primary adult mammalian cardiomyocytes are thought to be incapable of cytokinesis.

In general, there is an inverse relationship between proliferation and differentiation (Studzinski and Harrison 1999 Int Rev Cytol 189: 1-58), and molecules that promote differentiation may also repress cell cycle re-entry. It has been shown that the signaling molecule p38 mitogen-activated protein (MAP) kinase (p38) induces cell cycle exit and differentiation of many cell types, including differentiation of P19 cells to cardiomyocytes. Activated p38 phosphorylates downstream signaling molecules important for cardiomyocyte differentiation and hypertrophy. Four different p38 isoforms have been identified. The main isoform expressed in the heart is p38α. p38β and p38γ are expressed at low levels, and p38δ is not expressed in heart (Wang et al. 1997; Liao et al. 2001; Liang and Molkentin 2003). The invention demonstrates that the effects of p38 on differentiation and proliferation are reversible.

The invention is based, in part, on the discovery that adult mammalian ventricular cardiomyocytes can divide. One important mechanism used by mammalian cardiomyocytes to control proliferation is p38 MAP kinase activity. Several lines of evidence support these conclusions. First, p38 regulates expression of genes required for mitosis in cardiomyocytes. Second, p38 activity is inversely correlated with cardiac growth during development, and its overexpression blocks proliferation of fetal cardiomyocytes. Third, activation of p38 in vivo by MKK3bE reduces BrdU incorporation in fetal cardiomyocytes. Fourth, p38α knockout increased cardiomyocyte mitoses in neonatal mice. Furthermore, inhibition of p38 in cultures of adult cardiomyocytes promotes cytokinesis. Finally, mitosis is associated with transient dedifferentiation of the contractile apparatus. Thus, our data indicate that p38 is a key negative regulator of cardiomyocyte proliferation and that postmitotic cells can divide.

The invention demonstrates that adult mammalian cardiomyocytes can be induced to divide. Transgenic overexpression of oncogenes or cell cycle promoters have led to cardiomyocyte proliferation in adult animals. In all cases, however, transgene expression began in fetal development when cardiomyocytes normally proliferate. In these studies it is possible that cardiomyocyte differentiation was altered by the transgene. Experiments trying to confirm the effect of these genes on proliferation in wildtype adult cardiomyocytes indicated that the adult cardiomyocytes could not proliferate. For example, de novo expression of c-myc in adult myocardium in vivo employing an inducible system (Xiao et al. 2001 Circ Res 89: 1122-9) or viral expression of cyclin D1 (Tamamori-Adachi et al. 2003 Circ Res 92: e12-9.) failed to induce cardiomyocyte cytokinesis. Likewise, overexpression of c-myc as well as serum stimulation in vitro did not result in adult cardiomyocyte division (Claycomb and Bradshaw 1983 Dev Biol 99: 331-7; Xiao et al. 2001 Circ Res 89: 1122-9). This invention demonstrates that cardiomyocytes isolated from 3 month old rats can be induced to divide in vitro. The advantage of this approach is that the identity of cardiomyocytes and the presence of cytokinesis can be clearly demonstrated using light microscopy and immunofluorescence staining. Several proteins induced cardiomyocyte proliferation, and we saw the greatest response with FGF1 coupled with p38 inhibitor.

Approximately 7.2% of adult cardiomyocytes re-entered the cell cycle as measured by Ki67 staining. These cells may represent a distinct cell population of adult cardiomyocytes. All analyzed cells were positive for Nkx2.5, tropomyosin and troponin T and had typical morphology of adult cardiomyocytes. None had the appearance of stem cells or fetal cardiomyocytes. The simplest interpretation of our data, therefore, is that adult cardiomyocytes can divide.

In p38α knockout hearts, BrdU incorporation was increased 20-fold, indicating that DNA synthesis in adult cardiomyocytes is enabled by the absence of p38. Our in vitro experiments suggest that p38 inhibition can enhance cardiomyocyte mitosis or cytokinesis. Moreover, specific growth factors, not present in vitro, may also be useful.

The microarray data and immunofluorescence studies show upregulation of cdc2, cdc25B, cyclin D, and cyclin B, all factors required for cell cycle progression. p38 can regulate cardiomyocyte proliferation by modulating important cell cycle factors. In one aspect, the invention provides a model for regulation of cardiomyocyte proliferation wherein FGF1 upregulated fetal cardiac genes induces dedifferentiation. This process was independent of p38. By contrast, p38 inhibition promoted FGF1-induced DNA synthesis (S phase). FGF1 regulated genes involved in apoptosis, and this effect was also enhanced by p38 inhibition. Finally, p38 activity prevented upregulation of factors required for karyokinesis and cytokinesis, confirming a role for p38 in G2/M checkpoint control. In addition, when p38 inhibitor was removed from culture media after induction of DNA synthesis, cardiomyocytes failed to progress through G2/M and cytokinesis (data not shown). Thus p38 inhibition is required for growth factor mediated induction of all phases of the cell cycle and substantially enhances the proliferative capacity of mammalian cardiomyocytes.

In another aspect of the invention, transgenic and/or pharmacologic p38 inhibition can be used to induce growth factor-mediated mammalian cardiac regeneration. The invention has implications for the treatment of cardiac diseases. Although significant advances have been made in the management of acute myocardial infarction, ischaemic heart disease is still the leading cause of death. The present invention provides methods of cardiac regeneration through cardiomyocyte proliferationan. This approach is appealing because mammalian heart growth during fetal development is mediated by cardiomyocyte proliferation and not through stem cells. This concept resembles liver regeneration that is based on the proliferation of differentiated hepatocytes. Similar to the heart, the majority of hepatocytes are tetraploid and previous studies have shown that diploid, tetraploid and octoploid hepatocytes have similar capacities to proliferate. Interestingly, liver regeneration is inversely correlated with p38 activity. In addition, EGR-1 deficient mice exhibiting impaired liver regeneration are characterised by increased p38 activity and inhibition of mitotic progression. Furthermore, we recently demonstrated that cardiac regeneration in zebrafish is achieved through cardiomyocyte proliferation. The mitotic index in this study was less than 0.5% in the wound area. Our results show a similar mitotic index (0.14%) for adult mammalian cardiomyocytes. Thus, this study suggests that mammalian cardiac regeneration might be possible.

In one aspect of the invention, p38 inhibitors can be used to increase proliferation and/or de-differentiation of postmitotic mammalian cardiomyocytes. SB203580 (4-[5-(4-Fluorophenyl)-2-[4-(methylsulfonyl)phenyl]-1H-imidazol-4-yl]pyridine) is a highly potent pyridinyl imidazole inhibitor of p38, p40, stress-activating protein kinase (SAPK), cytokine suppression binding protein (CSBP) or reactivating kinase (RK). SB203580 inhibits p38α, β and β2 by competing with the substrate ATP. While SB203580 inhibits p38 activity, it does not significantly affect the activation of p38. SB203580 does not inhibit PKA, PKC, MEKs, MEKKs or ERK and JNK MAP kinases. SB202474 is an inactive analogue which is commonly used as a negative control of p38 MAP kinase inhibitor. SB239063 (trans-1-(4-Hydroxycyclohexyl)-4-(fluorophenyl)-5-(2-methoxypyrimidin-4-yl)imidazole) is a potent, cell permeable inhibitor of p38 MAP kinase which has been shown to inhibits IL-1 and TNF-β production in LPS-stimulated human peripheral blood monocytes. Many commercially available p38 inhibitors are pyridinyl imidazoles. For descriptions of additional p38 inhibitors see, for example, U.S. Pat. No. 6,093,742 and US Pub. No. 2004/0176325, which are herein incorporated by reference.

### p38 Inhibitors

A wide variety of p38 inhibitors can be useful in the present invention. Nine general classes of compounds are particularly noteworthy. Each of these classes of compounds should be understood to also encompass all pharmaceutically acceptable derivatives and can be used in association with one or more pharmaceutically acceptable excipients, diluents or carriers.
A. Derivatives of Nicotinic Acid Generally according to the Formula: 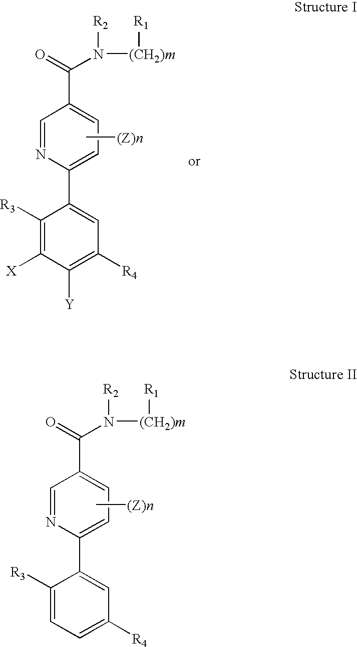
wherein:

R₁ is selected from the groups hydrogen, C₁₋₆alkyl which may be optionally substituted by up to three groups selected from C₁₋₆alkoxy, hydroxy, and halogen, C₂₋₆alkenyl, C₃₋₇cycloalkyl optionally substituted by one or more C₁₋₆alkyl groups, substituted and unsubstituted heteroaryl, substituted and unsubstituted phenyl;

R₂ is selected from hydrogen, C₁₋₆alkyl, and —(CH2)_{q}-C₃₋₇cycloalkyl optionally substituted by one or more C₁₋₆alkyl groups,

or —(CH2)ₘ-R₁ and R₂, together with the nitrogen atom to which they are bound form a four to six membered heterocyclic ring optionally substituted by up to three groups C₁₋₆alkyl groups;

R₃ is chloro or methyl;

R₄ is the group —NH—C(O)—R, —C(O)—NH—(CH2)ₐ-R′ wherein when a is 0 to 2, R′ is selected from hydrogen and C₁₋₆alkyl, substituted or unsubstituted C₃₋₇ cycloalkyl, substituted and unsubstituted phenyl, substituted and unsubstituted heteroaryl and substituted and unsubstituted heterocyclyl;

X and Y are each independently selected from hydrogen, methyl and halogen;

Z is halogen;

m is selected from 0, 1, 2, 3 and 4, wherein each carbon atom of the resulting carbon chain may be optionally substituted with up to two groups selected independently from C1-C6 alkyl and halogen; and

n is selected from 0, 1 and 2;
B. Substituted Biphenyl Amides Generally according to the Formula:

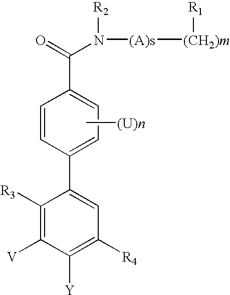

wherein A is a bond or a phenyl ring optionally substituted;

R₁ is selected form the groups hydrogen, C₁₋₆alkyl optionally substituted by one to three groups selected from oxo, cyano, and sulfoxide, C₃₋₇cycloalkyl optionally substituted by up to three groups independently selected from oxo, cyano, —S(O)ₚR₄, OH, halogen, C₁₋₆alkoxy, substituted and unsubstituted amines, substituted and unsubstituted amides, esters, substituted and unsubstituted sulfonamides; substituted and unsubstituted five to sevene membered heterocyclic ring, substituted and unsubstituted five to sevene membered heteroaryl ring, substituted and unsubstituted five to sevene membered bicyclic ring, and substituted and unsubstituted phenyl group;

R₂ is selected from hydrogen, C₁₋₆alkyl, and —(CH2)_{q}-C₃₋₇cycloalkyl optionally substituted by one or more C₁₋₆alkyl groups,

or —CH2)ₘ-R₁ and R₂, together with the nitrogen atom to which they are bound form a four to six membered heterocyclic ring containing one or two additional heteroatoms independently selected from oxygen, sulfur, and NH—R₇, wherein the ring is optionally substituted by one or two groups independently selected from oxo, C₁₋₆alkyl, halogen and trifluoromethyl;

R₃ is chloro or methyl;

R₄ is the group —NH—C(O)—R, —C(O)—NH—(CH₂)ₐ—R′; wherein:

R is selected from hydrogen and C₁₋₆alkyl, C₁₋₆alkoxy, substituted and unsubstituted —CH₂)-phenyl, substituted and unsubstituted —CH₂)-heteroaryl and substituted and unsubstituted —CH₂)-heterocyclyl, and substituted or unsubstituted —CH₂)—C₃₋₇ cycloalkyl;

and when a is 0 to 2,

R′ is selected from hydrogen and C₁₋₆alkyl, substituted or unsubstituted C₃₋₇ cycloalkyl, substituted and unsubstituted phenyl, substituted and unsubstituted heteroaryl and substituted and unsubstituted heterocyclyl, hydroxide, substituted and unsubstituted amines, substituted and unsubstituted amides; or

R₄ is a substituted or unsubstituted heterocycle, containing 1, 2, or 3 heteroatoms, taken from nitrogen, oxygen, sulfur and may contain one or two double bonds, wherein said double bonds could make the heterocycle aromatic, and the group 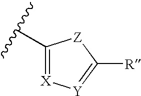
wherein

X and Y are each nitrogen and Z is oxygen,

X, Y and Z are each independently selected from nitrogen, oxygen, sulfur;

R″ is selected from hydrogen and C1-C4alkyl;

V and Y are each independently selected from hydrogen, methyl and halogen;

U is selected from methyl and halogen;

m is selected from 0, 1, 2, 3 and 4, wherein each carbon atom of the resulting carbon chain may be optionally substituted with up to two groups selected independently from C₁₋₆alkyl wherein the C₁₋₆alkyl group is optionally substituted by up to three hydroxy groups and wherein in some embodiments the sum of m+n is from 0 to 4;

n is selected from 0, 1 and 2;
C. Substituted pyrrolo[2.3-d]pyrimidin-4-yl Compounds Generally according to the Formula

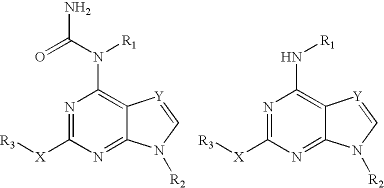

wherein R₁ is hydrogen, C₁₋₁₀alkyl, C₃₋₇cycloalkyl, C₃₋₇cycloalkylalkyl, C₅₋₇cycloalkenyl, C₅₋₇, cycloalkenylalkyl, aryl, arylalkyl, heterocyclic, heterocyclicalkyl, heteroaryl, or heteroarylalkyl moiety, all of the moieties may be optionally substituted;

R₂ is C₁₋₁₀alkyl, C₃₋₇cycloalkyl, C₃₋₇cycloalkylalkyl, C₅₋₇cycloalkenyl, C₅-₇ cycloalkenylalkyl, aryl, aryl-C₁₋₁₀alkyl, heteroaryl, heteroaryl-C₁₋₁₀alkyl heterocyclic, or heterocyclic-C₁₋₁₀alkyl moiety, all of the moieties may be optionally substituted;

X is a bond, O, N, or S;

R₃ is an optionally substituted aryl or optionally substituted heteroaryl moiety;

Y is carbon or nitrogen;
D. Fused Heteroaryl Derivatives Generally according to the Formula: 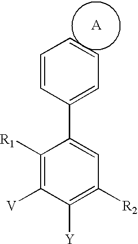
wherein:

A is a fused 5-membered heteroaryl ring substituted by —(CH₂)ₘ hetercyclyl wherein the heterocyclyl is a 5- or 6-memered heterocyclic ring containing one or two heteroatoms independently selected from oxygen, sulfur, and nitrogen optionally substituted by up to two substituents independently selected from oxo, C₁₋₆alkyl, —(CH₂)ₙphenyl, ether, keto, substituted or unsubstituted amine, substituted or unsubstituted amide; or

A is optionally further substituted by one substituent selected from ether, halogen, trifluoromethyl, —CN, ester, and C₁₋₆alkyl optionally substituted by OH;

R₁ is selected form methyl and chloro;

R₂ is selected from —C(O)—NH—(CH₂)_{q}—R′ or —NH—C(O)—R;

X and Y are each independently selected from hydrogen, methyl and halogen;

m and q are independently selected from 0, 1, and 2;

n is selected from 0, and 1

with the proviso that:

A is not substituted by —(CH₂)ₘNR₁₄R₁₅ wherein R₁₄ and R₁₅, together with the nitrogen to which they are bound form a five or six membered heterocyclic ring optionally containing one additional heteroatom selected from oxygen, sulfur, and N—R₁₆, wherein R₁₆ is selected from hydrogen or methyl;

when m is 0, the —CH₂)ₘ heterocyclyl group is not a 5- or 6-membered hetero cyclyl ring containing nitrogen optionally substituted by C1-C2alkyl, or —(CH₂)nCOOR
E. Substituted 2-phenyl-5-carboxamide pyridine-N-oxides Generally According to the Formula: 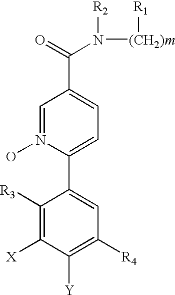
wherein:

R₁ is selected form the groups hydrogen, C₁₋₆alkyl optionally substituted by up to three groups independently selected from C₁₋₆alkoxy, OH and halogen, C₂₋₆alkenyl, —C₃₋₇cycloalkyl optionally substituted by or more C₁₋₆alkyl groups, substituted or unsubstituted phenyl group, and substituted or unsubstituted heteroaryl group;

R₂ is selected form hydrogen, C₁₋₆alkyl and —(CH2)_{q}-C₃₋₇cycloalkyl optionally substituted by or more C₁₋₆alkyl groups,

or —CH2)ₘ-R1 and R2, together with the nitrogen atom to which they are bound form a four to six membered heterocyclic ring optionally substituted by up to three C1-C6 alkyl groups;

R₃ is chloro or methyl;

R₄ is the group —C(O)—NH—(CH2)_{q}-R′ or —NH—C(O)—R;

X and Y are each independently selected from hydrogen, methyl and halogen;

m is selected from 0, 1, 2, 3 and 4, wherein each carbon atom of the resulting carbon chain may be optionally substituted with up to two groups selected independently from C1-C6 alkyl and halogen;

q is selected from 0, 1, and 2;

Within this class, the following compounds may be particularly useful: 6-{5-[(cyclopropylamino)carbonyl]-3-fluoro-2-methylphenyl}-N-(2,2-dimethylpropyl)-3-pyridinecarboxamide 1-oxide; 6-{5-[(cyclopropylamino)carbonyl]-3-fluoro-2-methylphenyl}-N-[(1R)-1,2,2-trimethylpropyl)]-3-pyridinecarboxamide 1-oxide; 6-{5-[(cyclopropylamino)carbonyl]-3-fluoro-2-methylphenyl}-N-(1,1-dimethylpropyl)-3-pyridinecarboxamide 1-oxide; 6-{5-[(cyclopropylamino)carbonyl]-3-fluoro-2-methylphenyl}-N-(1-ethylpropyl)-3-pyridinecarboxamide 1-oxide; 6-{5-[(cyclopropylamino)carbonyl]-3-fluoro-2-methylphenyl}-N-[(1S)-1,2,2-trimethylpropyl)]-3-pyridinecarboxamide 1-oxide; 6-{5-[(cyclopropylamino)carbonyl]-3-fluoro-2-methylphenyl}-N-[(1R)-1,2-trimethylpropyl)]-3-pyridinecarboxamide 1-oxide; 6-{5-[(cyclopropylamino)carbonyl]-3-fluoro-2-methylphenyl}-N-[(1S)-1,2-trimethylpropyl)]-3-pyridinecarboxamide 1-oxide; 6-{5-[(cyclopropylamino)carbonyl]-3-fluoro-2-methylphenyl}-N-[(3,4-dimethylphenyl)methyl]-3-pyridinecarboxamide 1-oxide;
F. Trisubstituted-8H-pyrido[2,3-d]pyrimidin-7-one Analogs Generally According to the Formula 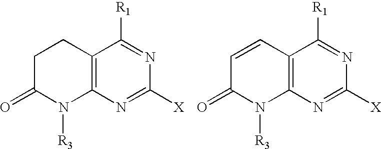
wherein:

R₁ is optionally substituted aryl or heteroaryl ring;

R₂ is selected from hydrogen, C₁₋₁₀alkyl, and C₃₋₇cycloalkyl, C₃₋₇cycloalkylalkyl, aryl, arylC₁₋₁₀alkyl, heteroaryl, heteroaryl C₁₋₁₀alkyl, heterocyclic, hetercyclic C₁₋₁₀alkyl moiety, which moieties may be optionally substituted or R₂ is the moiety X₁(CRR′)_{q} C(A₁)(A₂)(A₃), C(A₁)(A₂)(A₃);

A₁ and A₂ are optionally substituted C₁₋₁₀alkyl;

A₃ is hydrogen or optionally substituted C₁₋₁₀alkyl

R₃ is selected from C₁₋₁₀alkyl, and C₃₋₇cycloalkyl, C₃₋₇cycloalkyl C₁₋₄alkyl, aryl, aryl C₁₋₁₀₁alkyl, heteroaryl, heteroaryl C_{1-10aryl}alkyl, heterocyclic, hetercyclic C_{1-10aryl}alkyl moiety, which moieties may be optionally substituted;

X is R₂, OR₂, S(O)ₘR₂, (CH₂)ₙN(R′)S(O)ₘR₂, (CH₂)ₙN(R′)C(O)ₘR₂, mono and di-substituted amine;

X₁ is a NR, O, sulfoxide, CR″R′″

m is 0, 1, 2;

q is 0, or an integer from 1, to 10;
G. Compounds Generally According to the Formula

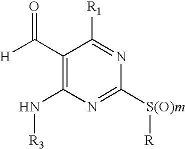

wherein R₁ is halogen, optionally substituted aryl or heteroaryl ring;

R₃ is selected from hydrogen, C₁₋₁₀alkyl, and C₃₋₇cycloalkyl, C₃₋₇cycloalkylalkyl, aryl, arylC₁₋₁₀alkyl, heteroaryl, heteroaryl C₁₋₁₀alkyl, heterocyclic, hetercyclic C₁₋₁₀alkyl moiety, which moieties may be optionally substituted, provided when R₃ is hydrogen R₁ is other than chlorine;

m is 0, 1, 2; and

R is C₁₋₄alkyl,
H. Substituted pyrimido[4,5-d]pyrimidin-2-one Derivatives Generally According to the Formula:

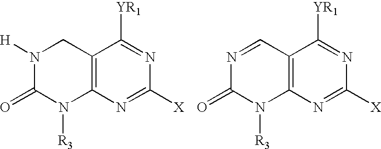

wherein R₁ is aryl or heteroaryl ring, which ring is optionally substituted;

R₂ is selected from hydrogen, C₁₋₁₀alkyl, and C₃₋₇cycloalkyl, C₃₋₇cycloalkylC₁₋₁1alkyl, aryl, arylC₁₋₁₀alkyl, heteroaryl, heteroaryl C₁₋₁₀alkyl, heterocyclic, hetercyclic C₁₋₁₀alkyl moiety, which moieties may be optionally substituted;

R₃ is selected from C₁₋₁₀alkyl, and C₃₋₇cycloalkyl, C₃₋₇cycloalkylC₁₋₁1alkyl, aryl, arylC₁₋₁₀alkyl, heteroaryl, heteroaryl C₁₋₁₀alkyl, heterocyclic, hetercyclic C₁₋₁₀alkyl moiety, which moieties may be optionally substituted; and

X is R₂, OR₂, S(O)ₘR₂, mono and di-substituted amine
9. Subtituted Triazole Analogs: 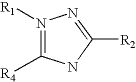
wherein:

R₁ is pyrid-4-yl, or pyrimidin-4-yl ring, which ring is optionally substituted one or more times with Y, C₁₋₄alkyl, C₁₋₄alkoxy, C₁₋₄alkylthio, C₁₋₄alkylsulfinyl, CH₂OR, mono and di-substituted amine, N-heterocycle ring, which ring is 5-, to 7-membered and optionally contains an additional heteroatom selected from oxygen, sulfur, NR′;

Y is X₁-Rₐ;

X₁ is sulfur NH or oxygen;

Rₐ is C₁₋₆alkyl, aryl, arylC₁₋₆alkyl, heterocyclic, heterocyclylC₁₋₆alkyl, heteroaryl, heteroarylC₁₋₆alkyl, wherein each of these moieties may be optionally substituted;

R₂ is hydrogen, substituted or unsubstituted C₁₋₁₀alkyl, substituted or unsubstituted alcohol, substituted or unsubstituted ester, substituted or unsubstituted C₁₋₁₀alkyl ether, substituted or unsubstituted sulfone, substituted or unsubstituted aryl ether, substituted or unsubstituted heteroaryl ether, substituted or unsubstituted heteroaryl C₁₋₁₀alkyl ether, substituted or unsubstituted heterocyclylC₁₋₁₀alkyl ether, substituted or unsubstituted heterocyclyl ether, substituted or unsubstituted C₃₋₇cycloalkyl ether moiety, wherein each of these moieties may be optionally substituted, halo-substituted C₁₋₁₀alkyl, C₂₋₁₀alkynyl, C₂₋₁₀alkynyl, substituted or unsubstituted C₃₋₇cycloalkyl, substituted or unsubstituted C₅₋₇cycloalkyl, aryl, substituted or unsubstituted heteroaryl, substituted or unsubstituted hetercyclyl;

R₄ is phenyl, naphtha-1-yl, naphtha-2-yl, or a heteroaryl which is optionally substituted by one or two substituents, each of which is independently selected from aryl, or fused bicyclic groups, and having substituents selected from substituted or unsubstituted amide, substituted or unsubstituted ester, keto group, substituted or unsubstituted sulfoxide, substituted or unsubstituted thioether, halogen, halo-C₁₋₆alkyl, cyano, nitro, ether, substituted or unsubstituted amine, substituted or unsubstituted sulfonamide;

### EXAMPLES

### Example 1

### De-Differentiation and Proliferation of Adult Cardiomyocytes

Animals, Cells, and Stimulation

Animal experiments were performed in accordance with guidelines of Children's Hospital, Boston and UCLA. Ventricular cardiomyocytes from fetal (E19), 2-day-old (P2) and adult (250-350 g) Wistar rats (Charles River) were isolated as described with minor modifications (Engel et al. 1999; Engel et al. 2003). After digestion of fetal or neonatal hearts (0.14 mg/ml collagenase II (Invitrogen), 0.55 mg/ml pancreatin (Sigma)) cells were cultured in DMEM/F12 (GIBCO) containing 3 mM Na-pyruvate, 0.2% BSA, 0.1 mM ascorbic acid (Sigma), 0.5% Insulin-Transferrin-Selenium (100×), penicillin (100 U/ml), streptomycin (100 μg/ml), and 2 mM L-glutamine (GIBCO). Adult cardiomyocytes were cultured for 1 day in standard medium (DMEM, 25 mM Hepes, 5 mM taurine, 5 mM creatine, 2 mM L-carnitine (Sigma), 20 U/ml insulin (GIBCO), 0.2% BSA, penicillin (100 U/ml), and streptomycin (100 μg/ml)). Cells were stimulated in culture medium without BSA containing 2 mM L-glutamine. Neonatal and adult cardiomyocytes were initially cultured for 48 h in the presence of 20 μM cytosine β-D-arabinofuranoside (araC, Sigma) and 5% horse serum before stimulation to prevent proliferation of non-myocytes. Adult cardiomyocytes were incubated another 3 days with araC during stimulation. Neonatal cardiomyocytes were stimulated every day with growth factors for BrdU and H3P analyses (FGF1 and NRG-1-1β at 50 ng/ml, IL-1β at 100 ng/ml, R&D Systems, all diluted in 0.1% BSA/PBS). SB203580 and LY294002 (Calbiochem) was added every day. Adult cardiomyocytes were stimulated with fresh medium and SB203580 every 3 days.

Transgenic Animals

The MKK3bE transgenic animals were reported previously (Liao et al. 2001. Proc Natl Acad Sci USA 98: 12283-8). p38α floxed allele was generated by homologous recombination in embryonic stem cells (Lexicon, Houston, Tex.) in which the first exon (containing ATG) was flanked by two loxP sites. See Supplemental Data for details. The floxed allele was bred into homozygosity and genotyped using Southern blot and PCR analysis. The conditional knockout was generated by crossing MLC-2a/Cre with homozygous floxed p38α mice. The MLC-2a/Cre mice contain CRE coding sequence knocked into MLC-2a allele. All transgenic animals were maintained in C57Black background. Only male animals were used for adult studies.

The p38α Mutant Mice

The p38α mutant mice were generated in collaboration with Lexicon Genetics, Inc. (The Woodlands, Tex.). The p38α conditional targeting vector was derived using the Lambda KOS system (Wattler et al. 1999). The Lambda KOS phage library, arrayed into 96 superpools, was screened by PCR using exon 1-specific primers (BI2-64: GAGGACCGCGGCGGG) and (BI2-65: CTTCCAGCGGCAGCAGCG). The PCRpositive phage superpools were plated and screened by filter hybridization using the 227 bp amplicon derived from primers BI2-64 and BI2-65 as a probe. The positive clones isolated from the library screen were further confirmed by sequence and restriction analysis. The 565 bp region containing Exon 1 of p38 α was first amplified by PCR using primers BI2-54: (CTCCTTGGAGCTGTTCTCGCG) and BI2-53: (ATGCAGGGCCACCCTGCTTGC) and cloned into pLF-Neo containing the flanking LoxP sites and an Frt-flanked Neo cassette. The final targeting vector was generated from this plasmid and the genomic DNA fragments from phage clones as illustrated in the FIG. 5. The Not I linearized targeting vector was electroporated into 129/SvEvBrd (Lex-1) ES cells. G418/FIAU resistant ES cell clones were isolated, and correctly targeted clones were identified and confirmed by Southern analysis using a 477 bp 5′-external probe (124/119), generated by PCR using primers (BI2-124: CATGCAGGGCTACTCTACC) and (BI2-119: GCCACCTTCAAGCATCTCC), and a 582 bp 3′-internal probe (138/141), amplified by PCR using primers (BI2-138: TAAGGGCCCAAAAGGTATGC) and (BI2-141: ACTGTCACCAGTAGAACAGC). Southern analysis using probe 124/119 detected a 7 Kb wildtype band and 9.4 Kb mutant band in Hind III digested genomic DNA while probe 138/141 detected a >11 Kb wild type band and >7.4 Kb mutant band in EcoRV digested genomic DNA. Two targeted ES cell clones were microinjected into C57BL/6 (albino) blastocysts. The resulting chimeras were mated to C57BL/6 (albino) females to generate mice that were heterozygous for the floxed p38α allele. They are further bred with Cre-expressing mouse line to generate homozygous p38α loxP/loxP and conditional p38 α!/! mice. Their genotype was determined by PCR using specific primer sets for cre (Cre-5: GCCACCAGCCAGCTATCAAC and Cre-3: GCTAATCGCCATCTTCCAGC), and p38a floxed and wildtype alleles (BI2-41: TCCTACGAGCGTCGGCAAGGTG and B12-125: AGTCCCCGAGAGTTCCTGCCTC). Wattler, S., M. Kelly, and M. Nehls. 1999. Construction of gene targeting vectors from lambda KOS genomic libraries. Biotechniques 26: 1150-6, 1158, 1160.

In Vivo BrdU Labeling

Pregnant MKK3bE (E21) and newborn p38α knockout mice (P3) were injected i.p. with 10 ml/kg body weight of BrdU (10 mM in saline) and sacrificed 18 h later. Adult mice (10 weeks) were injected with BrdU solution 96 h and 48 h before tissue collection. Neonatal hearts were fixed in ice-cold 10% buffered formalin, incubated in 30% sucrose (both over night at 4° C.), embedded in tissue freezing medium (Fisher), stored for 24 h at −20° C. and sectioned (10 μm, Leica 3050S). Adult hearts were embedded in tissue freezing medium (Fisher) without fixation.

Heart Growth

Images of hearts were analyzed with NIH Image 1.62 software to determine the maximal area (ma). Heart growth was calculated as (ma_{Ex}/ma_{Ex-1})*100-100, where Ex=specific embryonic day.

Immunofluorescence Staining

Staining was performed as described (Supplemental Table S3) (Engel et al. 1999; Engel et al. 2003). Immune complexes were detected with ALEXA 350, ALEXA 488 or ALEXA 594-conjugated secondary antibodies (1:200, Molecular Probes). DNA was visualised with DAPI (4′,6′-diamidino-2-phenylindole, 0.5 μg/ml, Sigma). For BrdU, cells were cultured in 30 μM BrdU, incubated after permeabilization for 90 min in 2N HCl/1% triton X-100 and washed 3 times in PBS.

p38 Kinase Assay and Western Blotting

p38 kinase activity was determined with the p38 MAP Kinase Assay kit (Cell Signaling). Hearts were homogenised in lysis buffer (10×tissue volume) containing 1 mM Pefabloc SC (Roche), sonicated, and centrifuged. Anti-phospho-p38 immunoprecipitates for kinase reactions were derived from 200 μg protein. Extracts containing 20 μg of protein or 20 μl of kinase reaction were resolved by NuPAGE Novex Bis-Tris Gels (Invitrogen) and detected as described (Supplemental Table S3). Signals were quantified by NIH Image 1.62 software.

Electroporation and Adenoviral Infection

Plasmids to overexpress p38α and p38αDN (Raingeaud et al. 1995 J Biol Chem 270: 7420-6) were electroporated into fetal cardiomyocytes according to manufacturer's instructions (Amaxa). Transfection efficiency of cardiomyocyte cultures was >30% (Gresch et al. 2004 Methods 33: 151-63). Neonatal cardiomyocyte cultures were infected with adenoviral constructs Ad-p38αDN, Ad-p38βDN (Wang et al. 1998. J Biol Chem 273: 2161-8) and Aδ-GFP (Clontech) after preplating. Infection efficiency of cardiomyocyte cultures was >90% as determined by indirect immunofluorescence.

Proliferation Assay

Cells were trypsinized, washed in ice-cold PBS, and cell number was determined with hemocytometer. Percentage of cardiomyocytes was determined as described (Engel et al. 1999 Circ Res 85: 294-301).

Microarray Analysis and RT-PCR

RNA of neonatal cardiomyocytes was prepared 72 h after stimulation using Trizol (Invitrogen). RT-PCR was performed following standard protocols (Supplemental Table S4). Affymetrix technology was applied using the Rat Expression Set 230.

Statistical Analysis

Eighteen to 40 hearts of 3 different litters were used for quantitative analyses of maximal areas. For immunofluorescence analyses 1,500 fetal or neonatal cardiomyocytes were counted. For adult cardiomyocyte analyses in vitro the following number of cells were counted: 500-2,000 for BrdU or Ki67, 9,000-25,000 for H3P, and 12,000-45,000 for aurora B. For in vivo MKK3bE and p38α knockout experiments 2 different litters were used. We counted 1,500-2,000 cells in each apex, left and right ventricle per heart. For adult experiments we analyzed 2 p38α^{Δ/Δ} and 2 p38^{lox/lox} hearts (24 sections each). Statistical significance was determined using Student's t test.

p38 Inhibition Regulates Genes Critical For Mitosis in Cardiomyocytes

To determine the effect of p38 inhibition on cardiomyocyte differentiation and proliferation, a specific inhibitor of p38α and p38β, SB203580, was used and evaluated using cDNA microarray analyses using neonatal rat cardiomyocytes. Known genes that were consistently up- or down-regulated 2-fold or more by p38 inhibition after 72 hours were grouped into functional classes and clustered by response (Supplemental Table S1). Expression changes of a subset of genes were validated by RT-PCR.

Downregulation of cyclin A is an early sign of cell cycle exit in mammalian cardiomyocytes. In addition, it has been shown that cardiac-specific overexpression of cyclin A2 from embryonic day 8 into adulthood increases cardiomyoctye mitosis during postnatal development. In one aspect of the invention, it was shown that p38 inhibition upregulated cyclin A2. p38 inhibition also regulated other genes involved in mitosis and cytokinesis, including cyclin B, cdc2, and aurora B. We expected that these changes might also be associated with evidence of dedifferentiation, such as induction of fetal genes. However, only a slight induction of ANP was observed. Thus, p38 activity regulates genes important for mitosis in cardiomyocytes.

Stimulation of neonatal cardiomyocytes with FGF1 induces fetal gene expression. To determine if FGF1, in combination with p38 inhibition, can reverse differentiation and induce cell cycle re-entry, we repeated cDNA microarray analyses (Supplemental Table S1). FGF1 upregulated genes that are associated with fetal cardiac development, including ANP and BNP, and the Ets-related transcription factor PEA3. In addition, FGF 1 upregulated genes previously implicated in regeneration and cell cycle control, including Mustang. Finally, FGF1 downregulated pro-apoptotic genes, like CABC1, and upregulated anti-apoptotic genes, like PEA15. Taken together, these data suggest that FGF1 induces partial dedifferentiation and protects cardiomyocytes from apoptosis.

Expression analysis revealed that p38 inhibition and FGF1 together modulate expression of specific genes, whereas p38 inhibition or FGF1 stimulation alone had less effect. For example, p38 inhibition and FGF1 dramatically modulated expression of the cytokinesis regulator Ect2, the bHLH factor SHARP1, the cell cycle regulated protein CRP1, and the mediator of ventricular cardiomyocyte differentiation, IRX4. For a subset of cell cycle-perpetuating factors, including Ki67, cdc2, and cyclin A, and the cell cycle inhibitor p27, the combined effect of p38 inhibition and FGF1 stimulation was even greater at the protein level. The proliferation marker Ki67 (Brown and Gatter 2002 Histopathology 40: 2-11), for example, was increased 7-fold. Finally, p38 inhibitor and FGF1, but neither factor alone, led to phosphorylation of Rb, a key cell cycle regulator. Taken together, our data indicate that p38 inhibition and FGF1 stimulation act synergistically to induce expression of genes involved in proliferation and regeneration.

p38 Activity Blocks Fetal Cardiomyocyte Proliferation

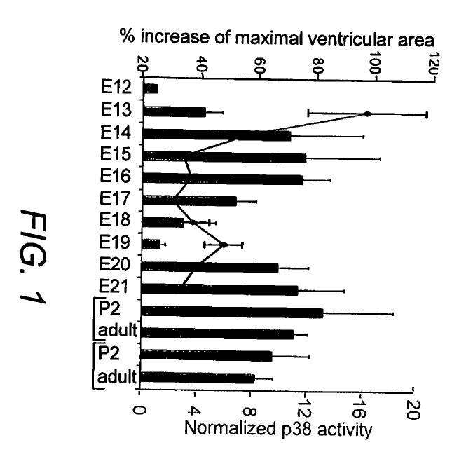

Fetal cardiomyocytes proliferate during development but lose this capacity shortly after birth. The switch from proliferative to hypertrophic growth has been associated with up- and downregulation of many factors. However, its mechanism is not understood. To determine if p38 regulates fetal cardiomyocyte proliferation, we examined prenatal cardiac growth. We collected rat hearts at sequential developmental stages (E12-E21, P2, and adult), and assessed the cardiac growth rate (n=18-40 per time point) and p38 activity (n=5 litter). Cardiac growth rate mediated predominantly by fetal cardiomyocyte proliferation was defined as the percentage increase of maximal ventricular area, as shown in FIG. 1. The rate of cardiac growth decreased sharply from E13 to E15 (p<0.01), accelerated from E17 to E19 (p<0.01), and decreased again. The p38 activity, by contrast, was inversely correlated with cardiac growth. The p38 activity was low at E12, peaked at E15, declined to a second low at E19, rose again and stayed high in adults (p<0.01). At E13, for example, cardiac area doubled and p38 activity was low (4.51). In contrast, at E15 cardiac area increased only 35% and p38 activity was high (11.89). These data indicate an association between p38 activity and fetal cardiomyocyte proliferation.

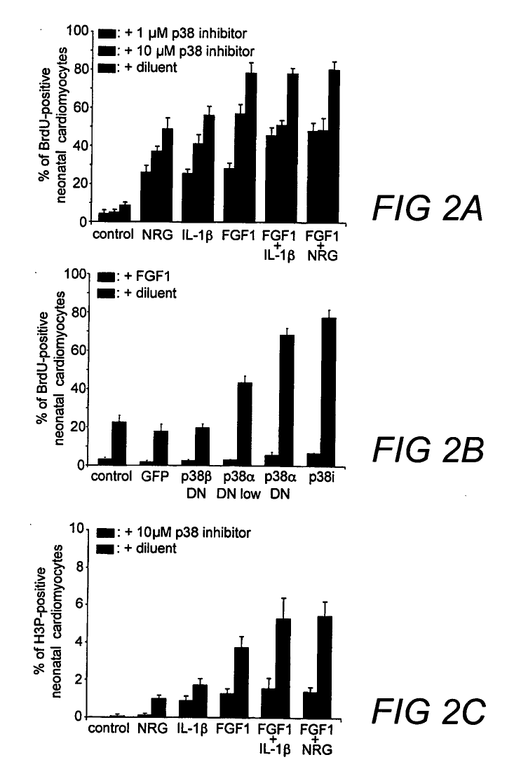

FIG. 2A-2C are graphs demonstrating that p38α regulates neonatal cardiomyocyte proliferation potential. To directly assess the role of p38 in regulating fetal cardiomyocyte proliferation, we overexpressed GFP, p38α and a dominant negative form of p38α (p38αDN) in fetal (E19) cardiomyocytes. The p38αDN is mutated in its dual phosphorylation site causing lack of kinase activity. Cells were electroporated, cultured for 36 hours, and stimulated for 24 hours with FGF1 in the presence of BrdU (5-bromo-2′-deoxyuridine), a marker of DNA synthesis. The rate of BrdU incorporation in mock-transfected cells (GFP) was 23±5.2%. Overexpression of p38α (3.4±1.9%), but not p38αDN (19.2±4.8%), decreased FGF1-induced BrdU incorporation significantly. The p38 activity is very low in the fetal heart at this stage of development, so overexpression of p38αDN was not expected to have a significant effect. These results indicate that p38α is a potent regulator of fetal cardiomyocyte proliferation in vitro.

To determine the role of p38 activation in vivo, we examined transgenic animals with cardiomyocyte-specific expression of a constitutively active upstream kinase for p38, MKK3bE. Targeted activation of p38 in ventricular myocytes was achieved in vivo by using a gene-switch transgenic strategy resulting in the expression of MKK3bE mutant protein under the control of the alpha MHC promoter. Previously, it has been demonstrated that activation of p38 kinase activity causes a thin ventricular wall. The underlying mechanism of this phenotype is unclear, but induction of apoptosis was excluded. BrdU incorporation in fetal cardiomyocytes (E21) was reduced from 18.2±3.4% to 15.0±2.9% in MKK3bE transgenic hearts. This is a reduction of 17.6% (p<0.05) in cardiomyocyte proliferation. In one aspect, the invention demonstrates that p38 activity is a potent negative regulator of fetal cardiomyocyte proliferation in vitro and in vivo.

p38α Inhibition Promotes Neonatal Cardiomyocyte Proliferation in vitro

Several growth factors have a limited capacity to induce DNA synthesis in neonatal cardiomyocytes, including FGF1 (Pasumarthi and Field 2002). We screened 45 extracellular factors at two different concentrations for their ability to induce BrdU incorporation in neonatal (P2) cardiomyocytes. Cells were stimulated every 24 hours for 3 days and pulse-labeled with BrdU for the final 24 hours. We confirmed previous studies showing that FGF1, IL-1β, and NRG-1-β1 are potent growth factors for neonatal cardiomyocytes (Supplemental Table S2) (Pasumarthi and Field 2002 Circ Res 90: 1044-54).

Inhibition of p38 activity by SB203580 increased BrdU incorporation 2.8-fold in neonatal cardiomyocytes stimulated with FGF1 (p<0.01). Similar results were obtained after stimulation with IL-1β and NRG-1-β1. Thus, inhibition of p38 activity augments growth factor-mediated DNA synthesis in neonatal cardiomyocytes.

To support the specificity of SB203580, we repeated these experiments with dominant negative forms of p38α (p38αDN) and p38β (p38βDN). Adenovirus-mediated expression of p38αDN was as effective as SB203580 in increasing growth factor-mediated BrdU incorporation. By contrast, expression of p38βDN had no effect on DNA synthesis. These results are consistent with previous findings showing that p38α and p38β have distinct downstream targets (Enslen et al. 1998; Wang et al. 1998). Taken together, our data indicate that the effect of p38 on DNA synthesis in neonatal cardiomyocytes is mediated by p38α.

To determine if p38 also regulates karyokinesis in neonatal cardiomyocytes, we assayed mitosis by immunofluorecence staining of phosphorylated histone-3 (H3P). Inhibition of p38 activity using SB203580 increased the number of H3P-positive cells 3.9-fold in the presence of FGF1+NRG-1-β1, resulting in 5.4±0.8% H3P-positive cardiomyocytes (p<0.01). This value is comparable to that of proliferating cell lines and the mitotic index of fetal cardiomyocytes during embryonic development (E12, 3.7±0.6%). Thus, p38 activity regulates neonatal cardiomyocyte karyokinesis.

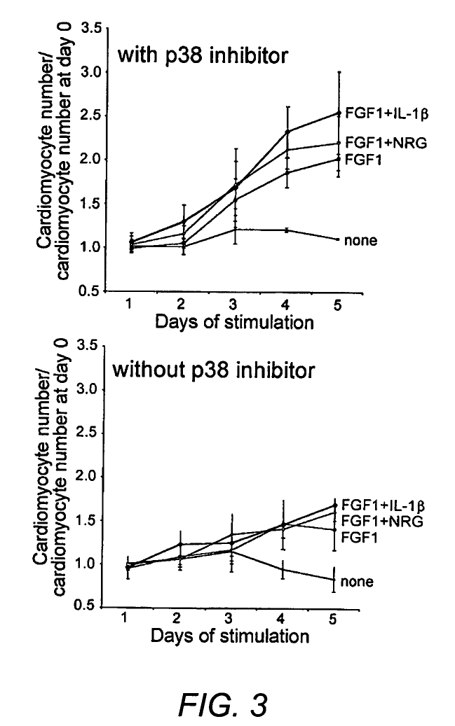

During postnatal development, mammalian cardiomyocytes frequently undergo karyokinesis without cytokinesis, and approximately 60% of human, and 85% of rat, adult cardiomyocytes are binucleated (Brodsky 1991 Cell Ploidy in the Mammalian Heart. Harwood Academic Publishers, New York). To test if p38 regulates cell division in neonatal cardiomyocytes, we performed cell count experiments. The percentage of cardiomyocytes was determined by tropomyosin staining and FACS analyses. Cells were incubated with SB203580 and stimulated once with growth factors on day 0. As shown in FIG. 3, this resulted in significantly increased cell numbers (day 3: p<0.05, day 4: and 5: p<0.01). The maximal increase in cardiomyocyte number of 2.6-fold was seen with FGF1+IL-1β stimulation at day 5. There was no evidence of binucleation by FACS analysis (data not shown).

To determine if neonatal cardiomyocytes can divide more than once, we stimulated cardiomyocytes continuously with FGF1 in the presence of SB203580 and monitored cell proliferation. The number of cardiomyocytes continued to increase until cells reached confluence. This indicates multiple rounds of cardiomyocyte division. BrdU and H3P analyses further supported that cardiomyocyte proliferation continued until cells became confluent. Thus, cardiomyocytes in the presence of p38 inhibition and growth factor stimulation continue to proliferate until mitosis is abrogated by contact inhibition.

To confirm that p38 inhibition promotes cardiomyocyte cell division, we assayed cytokinesis using immunofluorescence staining with aurora B or survivin antibodies. Aurora B kinases form a complex with inner centromere protein and survivin. Both proteins associate with centromeric heterochromatin early in mitosis, transfer to the central spindle, and finally localise to the contractile ring and midbody (Wheatley et al. 2001). Thus, aurora B and survivin are markers of cytokinesis. Aurora B and survivin assays confirmed that p38 inhibition and growth factor stimulation induced neonatal cardiomyocyte cytokinesis in vitro.

Increased Cardiomyocyte Mitosis in p38α Knockout Mice

To determine if proliferation of neonatal cardiomyocytes can be modulated by p38α inhibition in vivo, we examined mice in which p38α activity was disrupted specifically in cardiomyocytes. The conditional knockout (p38α^{Δ/Δ}) was achieved by crossing homozygous floxed p38α mice (p38^{loxP/loxP}) with a cardiomyocyte-specific cre line (MLC-2a/Cre). Western analyses indicated a dramatic reduction (>90%) of p38α protein specifically in cardiomyocytes. p38β and p38γ protein levels were unaffected. Cardiac-specific deletion of p38α diminished p38α downstream signaling (MAPKAPK2) but did not affect ERK phosphorylation.

To analyze the effect of p38α inactivation on the cell cycle in neonatal cardiomyocytes in vivo, we assayed BrdU and H3P in p38α^{Δ/Δ} mice. Among littermates, BrdU incorporation was highest in p38α^{Δ/Δ} mice. BrdU incorporation in neonatal cardiomyocytes (P4) was increased from 14.2±2.0% to 17.2±3.1% (17.2% increase, p<0.05). These data indicate that reduced p38α protein causes increased cardiomyocyte DNA synthesis in vivo. H3 phosphorylation was increased from 0.13±0.05% to 0.25±0.07% (92.3% increase, p<0.01) indicating that reduced p38α protein resulted in increased mitosis in cardiomyocytes in vivo.

Furthermore, we examined the effects of p38α protein reduction on BrdU incorporation in adult cardiomyocytes. To distinguish between adult cardiomyocytes and interstitial cells, hearts were sectioned and stained for the cardiac transcription factor GATA4 and a marker for cell membranes, Caveolin. We detected BrdU-positive adult cardiomyocytes in vivo. The number of BrdU-positive cardiomyocytes per longitudinal section in p38α^{Δ/Δ} mice (1.7±0.4) was 20-fold greater than observed in p38^{loxP/loxP} mice (0.08±0). Taken together, our data indicate that p38α is a negative regulator of cardiomyocyte proliferation in vivo.

Adult Cardiomyocytes Divide

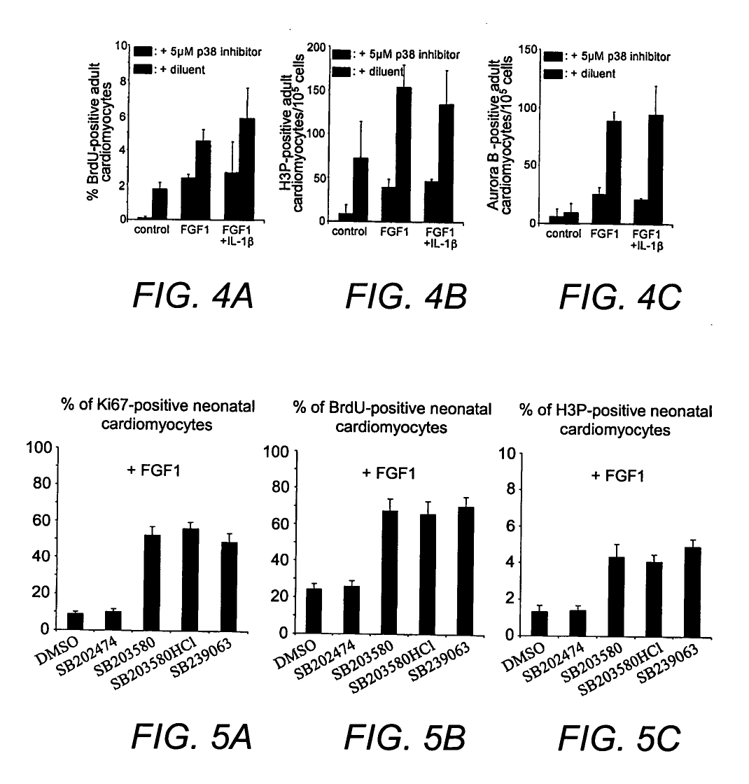

In contrast to neonatal cardiomyocytes, previous studies indicate that no DNA synthesis, karyokinesis or cytokinesis occurs in rat cardiomyocytes three weeks after birth (Rumyantsev 1977 Int Rev Cytol 51: 186-273; Pasumarthi and Field 2002 Circ Res 90: 1044-54). To determine if p38 inhibition promotes growth factor-mediated DNA synthesis in adult cardiomyocytes, we repeated cell proliferation assays using ventricular cardiomyocytes from 12-week old rats. As an additional cardiomyocyte-specific marker we employed the transcription factor Nkx2.5. Cardiomyocytes were isolated at day 0, and allowed to recover for 24 hours. Cells were then stimulated every three days with growth factors in the presence or absence of SB203580 for 12 days and assayed for BrdU. FGF1 alone and FGF1+IL-1β induced BrdU incorporation in more than 2% of adult cardiomyocytes. Inhibition of p38 doubled the effect of growth factors (p<0.01, FIG. 4A). These data demonstrate that p38 inhibition promotes growth factor-induced DNA synthesis in adult cardiomyocytes.

To determine if adult cardiomyocytes can undergo karyokinesis, we performed H3P analyses. Inhibition of p38 activity increased the number of H3P-positive cardiomyocytes 3.7-fold in the presence of FGF1 (p<0.01, FIG. 4B). These findings indicate that p38 regulates karyokinesis of adult cardiomyocytes.

To learn if adult mammalian cardiomyocytes can undergo cytokinesis we assayed aurora B. Inhibition of p38 increased cytokinesis 3.8-fold (p<0.01, FIG. 4C). The maximum effect was observed with p38 inhibition and FGF1. Although most proliferating adult cardiomyocytes were mononucleated, we also observed binucleated cells undergoing cytokinesis. These data indicate that adult ventricular cardiomyocytes can divide.

To estimate how many cardiomyocytes proliferate after 12 days of stimulation, we repeated these experiments using Ki67. In neonatal cardiomyocytes, FGF1 induced DNA synthesis, but failed to induce proliferation and Ki67 expression. By contrast, FGF1 stimulation in the presence of SB203580 resulted in both cardiomyocyte proliferation and Ki67 expression. Thus, Ki67 is an excellent marker for cardiomyocyte proliferation. In adult cardiomyocytes, stimulation with FGF1 alone resulted in 1.7±0.5% Ki67-positive cells (data not shown). However, stimulation with FGF1 and p38 inhibitor resulted in 7.2±1.2% Ki67-positive adult cardiomyocytes (p<0.01). Taken together, these data indicate that adult cardiomyocytes can proliferate in vitro, and that p38 potently controls this process.

Sarcomeres Dedifferentiate During Cardiomyocyte Proliferation

Fetal cardiomyocytes transiently dedifferentiate during mitosis in vivo. To learn if growth factor stimulation and p38 inhibition induce sarcomeric dedifferentiation in adult cardiomyocytes, we examined 100,000 stimulated cells using troponin T and tropomyosin antibodies. We observed 146 adult cardiomyocytes in mitosis. All non-mitotic adult cardiomyocytes had a striated sarcomeric structure with distinct Z-discs that was maintained during prophase (n=68). During prometaphase, however, adult cardiomyocytes lost Z-discs and all cells in metaphase and anaphase (n=78) showed absent Z-discs. In addition, a mesh of tropomyosin was formed around the chromosomes. In metaphase, this mesh became a ring. In telophase, sarcomeric striations began to be restored. Thus, mitosis in adult cardiomyocytes is associated with transient dedifferentiation of the contractile apparatus, a process similar to that observed in proliferating fetal cardiomyocytes in vivo. In addition, aurora B staining showed adult cardiomyocytes in early and late phases of cytokinesis. These findings indicate the formation of a contractile ring, cleavage furrow and midbody in dividing cardiomyocytes. Finally, the break of the midbody resulted in two spreading daughter cells containing an aurora B-positive remnant. These data suggest that proliferating adult cardiomyocytes dedifferentiate and then divide into new functional cardiomyocytes with differentiated sarcomeres.

Role of p38 in Cardiomyocyte Proliferation

Our microarray and proliferation data demonstrated that p38 inhibition promotes induction of DNA synthesis and G2/M transition in cardiomyocytes. However, inhibition of p38 alone had little or no effect on DNA synthesis or mitosis, suggesting that p38 and growth factors act sequentially to control progression through the different cell cycle phases. The fact that p38 inhibition can promote induction of DNA synthesis suggested that p38 and growth factors also act synergistically to control cardiomyocyte proliferation. To find a molecular explanation for this synergy, we re-examined our cDNA microarray data. We discovered that p38 inhibition downregulated Seta/Ruk, an adaptor protein that binds and inhibits PI3 kinase (Gout et al. 2000). Moreover, we found that Akt, a downstream target of PI3 kinase, is significantly phosphorylated in p38α knockout mice. To determine if PI3 kinase is required for FGF1 signaling in cardiomyocytes, we used the specific PI3 kinase inhibitor LY294002 (10 μM) (Vlahos et al. 1994). LY294002 abolished FGF1-induced DNA synthesis, suggesting that this process may require PI3 kinase activity. Thus, p38 inhibition may act synergistically with growth factors by downregulating antagonists of PI3 kinase.

The above results suggest a model for cardiomyocyte proliferation: p38 inhibits the transition from S phase to mitosis by downregulating mitotic genes. p38 inhibition acts synergistically with FGF1 to promote cell cycle progression, possibly through molecules like PI3 kinase.

### Example 2

### In Vivo Effects of p38 Inhibitors Following Myocardial Infarct

The effects of a variety of p38 inhibitors on adult rat cardiomyocytes were compared using Ki67, BrdU, and H3P (FIG. 5A shows the percentage of Ki67-positive neonatal cardiomyocytes. FIG. 5B shows the percentage of BrdU-positive neonatal cardiomyocytes and (FIG. 5C shows the percentage of H3P-positive neonatal cardiomyocytes). The compounds tested in FIGS. 5A-5C include SB203580, which has 100- to 500-fold selectivity over GSK3β and PKBα, SB203580 HCL (water insoluble), SB202474, a negative control commonly use for MAP kinase inhibition studies, and SB239063 which has >200-fold selectivity over ERK and JNK.

The p38 inhibitors were tested for in vivo effect following myocardial infarct. For the evaluation of left ventricular function, transthoracic echocardiogram can be performed on the rats after myocardial infarction 1 day or 14 days right. Rats can be anesthetized with 4-5% isoflurane in an induction chamber. The chest can be shaved, and the rats can be placed in dorsal decubitus position and intubated for continuous ventilation. 1-2% isoflurane can be continuously supplied via a mask. 3 electrodes can be adhered to their paws to record the electrocardiographic tracing simultaneously with the cardiac image identifying the phase of a cardiac cycle.

Echocardiograms can be performed with a commercially available echocardiography system equipped with 7.5 MHz phased-array transducer (Philips-Hewlett-Packard). The transducer can be positioned on the left anterior side of the chest. Longitudinal images of the heart can be obtained, including the left ventricle, atrium, the mitral valve and the aorta, followed by the cross-sectional images from the plane of the base to the left ventricular apical region. M-mode tracings can be obtained at the level below the tip of the mitral valve leaflets at the level of the papillary muscles. Fractional shorting (FS) can be calculated as a measure of systolic function, according to the M-mode tracing from the cross-sectional view: maximal LV end-diastolic diameter (at the time of maximal cavity dimension), minimal LV end-systolic diameter (at the time of maximum anterior motion of the posterior wall), FS (%)={(LVEDD−LVESD)/LVEDD}×100.

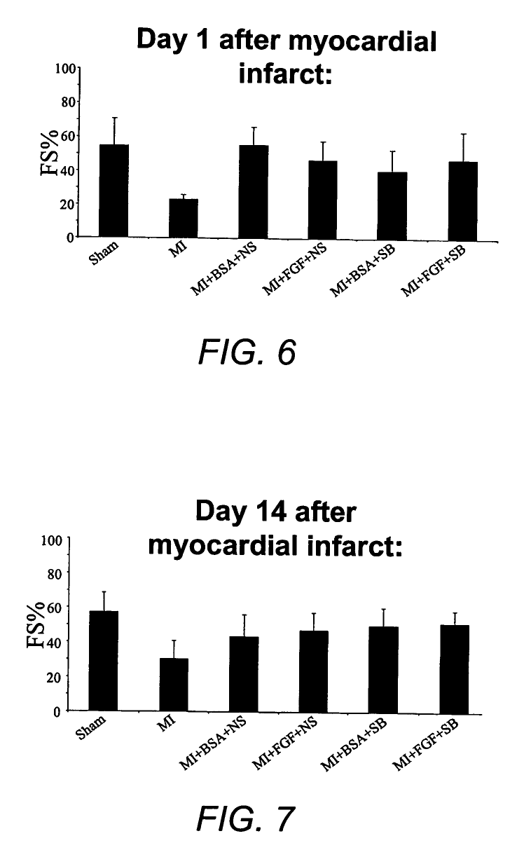

FIG. 6 demonstrates the effect of a p38 inhibitor (SB203580) with or without FGF on fractional shorting (FS) as a measure of systolic function one day after myocardial infarct. The sham-operated animals showed no significant changes in FS. The control (MI) showed a decrease in FS after myocardial infarct. However, the decrease in FS was significantly reduced when p38 inhibitor was given. FIG. 7 demonstrates the effect of a p38 inhibitor (SB203580) with or without FGF on fractional shorting (FS) 14 days after myocardial infarct. NS indicates a control with normal saline instead of the p38 inhibitor.

### Example 3

### Further In Vivo Effects of p38 Inhibitors Following Myocardial Infarct

To determine whether p38 inhibition/FGF1 stimulation can induce cardiomyocyte proliferation in vivo and whether it has a positive effect on cardiac function after cardiac injury we created myocardial infarctions (MI) in adult rats (250 g) by coronary artery ligation. The p38 inhibitor SB203580 HCl or its vehicle, saline, were injected intraperitoneal every three days for the first month of the study. FGF1 or its carrier BSA was injected mixed with self-assembling peptides once into the infarct border zone immediately after coronary artery ligation. We injecting a total of 80 μl of 400 ng/ml FGF1, given at 3 different injection sites, into 400 mg of infarcted myocardium estimated to deliver a FGF 1 concentration to the cardiomyocytes of approximately 50 to 100 ng/ml. Animals were analyzed 24 hours, 2 weeks, and 3 month after surgery. We performed two blinded and randomized studies using 62 rats for the 2 week and 61 rats for the 3 month experiment, with at least 10 animals in each experimental group. Animals were treated with saline plus BSA (control), SB203580 HCl plus BSA (p38i), saline plus FGF1 (FGF1), or SB203580 HCl plus FGF1 (p38i/FGF1).

p38 Inhibition Enables Cardiomyocyte Proliferation In Vivo After MI

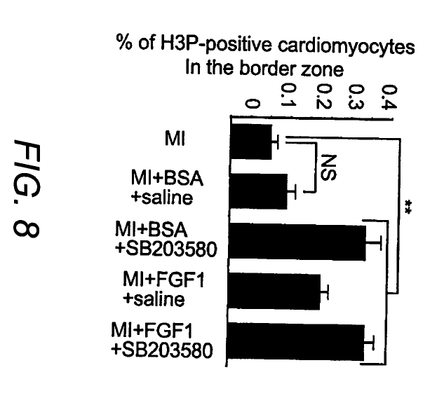

To determine whether p38 inhibition/FGF1 stimulation can induce cardiomyocyte proliferation we the mitosis marker H3P at two levels of sections. Histone 3 phosphorylation in cardiomyocytes were significantly increased in animals treated with FGF1/p38i. Interestingly, p38 inhibition alone could in contrast to our in vitro study also enhance cardiomyocyte mitosis. This is probably due to the fact that the heart releases a variety of growth factors during infarction. Our previous data revealed that p38 inhibition can induce cardiomyocyte proliferation with a variety of different growth factors. Taken together, our data indicate that p38 inhibition can increase cardiomyocyte proliferation in vivo (FIG. 8).

FGF1/p38 Inhibitor Treatment Improve Heart Function After MI

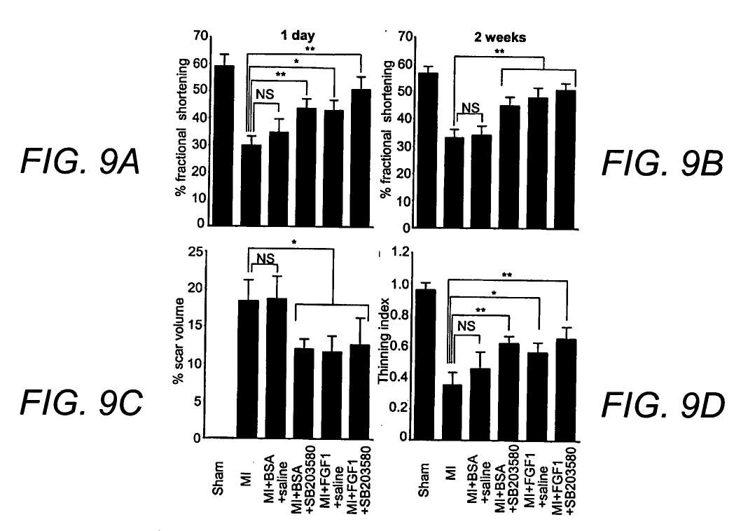

To determine whether p38 inhibition/FGF1 stimulation has a positive effect on cardiac function after cardiac injury we determined fractional shortening, scar volume, and wall thinning. Twenty-four hours after MI, left ventricular fractional shortening decreased as anticipated compared with sham-operated myocardium, and injection of saline and BSA did not significantly improve fractional shortening. However, in infarcted hearts with injection of FGF1 and/or p38i fractional shortening was significantly improved (FIG. 9A). At day 14 after infarction, improvement of fractional shortening was maintained in hearts that received SB203580 HCl, FGF1 or FGF1+SB203580 HCl (FIG. 9B). Taken together, these data demonstrate all treatments prevent impairment of ventricular function after cardiac injury.

Myocardial infarction disturbs loading conditions within the heart, causes ischemic and oxidative stresses, and activates various local and systemic neurohormonal systems (Pfeffer and Braunwald, 1990). These alterations to the extracellular environment trigger left ventricular (LV) remodeling characterized by necrosis and thinning of the infarcted myocardium, LV chamber dilation, fibrosis both at the site of infarct and in the non-infarcted myocardium, and hypertrophy of viable cardiomyocytes. Early remodeling may be adaptive and sustain LV function in the short term, however persistent remodeling contributes to functional decompensation and eventually the development of the clinical syndrome of heart failure (Swynghedauw, 1999). Therefore, improved heart function can be achieved through several mechanisms.

To determine if p38 inhibition and FGF1 stimulation have an effect on infarct size we determined scar volume using trichrome stain. Quantification of scar volume revealed that the scar size at 2 weeks was significantly reduced in all rats treated with p38i and/or FGF1 (FIG. 9C).

Ventricular wall thinning is an important parameter of heart function. Thus, we determined the thickness of the ventricular wall after injury. For this purpose we calculated the thinning index (ratio of minimal ventricular wall thickness to maximal thickness of the septum). Quantification of thinning index revealed that left ventricular wall thinning was significantly reduced in all rats treated with p38i and/or FGF1 (FIG. 9D).

FGF1/p38 Inhibitor Improved Heart Function Permanently

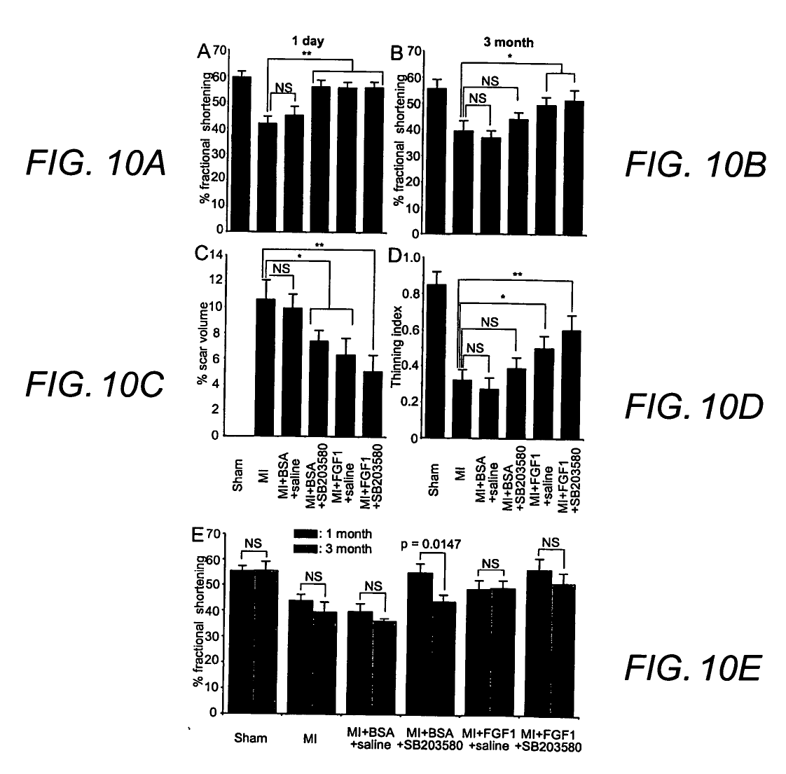

Next, we wondered if the observed effect is maintained over time and whether heart functions stays improved after ending therapy. As shown in our first experiment, twenty-four hours after MI, left ventricular fractional shortening decreased as anticipated compared with sham-operated myocardium, and fractional shortening was significantly improved in infarcted hearts with injection of FGF1 and/or p38i (FIG. 10A). At 3 month after infarction, improvement of fractional shortening was maintained in hearts that received FGF1 and/or SB203580 HCl (FIG. 10B). However, injection with p38 inhibitor alone shows no improved fractional shortening. It appears that after ending SB203580 injection at 2 month fractional shortening is decreasing over time (FIG. 10E). Taken together, these data demonstrate that FGF1 stimulation with or without p38 inhibition prevent impairment of ventricular function after cardiac injury.

To determine if p38 inhibition and FGF1 stimulation have an long-term effect on infarct size we determined scar volume using trichrome stain. Quantification of scar volume revealed that the scar size at 3 month was significantly reduced in all rats treated with p38i and/or FGF1 (FIG. 10C).

Ventricular wall thinning however, was again only significantly improved after FGF1 with or without p38 inhibition (FIG. 10D).

FGF1/p38 Inhibitor Treatment Increases Vascularization

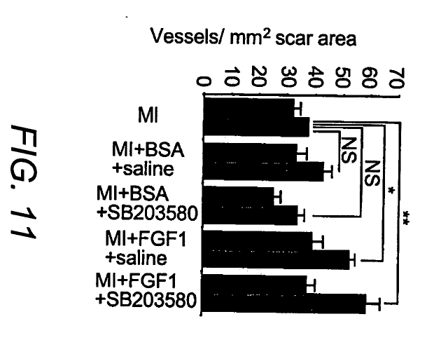

All data show a clear trend that the combination of p38 inhibitor together with FGF1 has the best positive effect on heart function after MI. One possible explanation is the angiogenic effect of FGF1. To determine the effect of our treatments on vascularization we determined the vessel density in the scar area. Vessels were visualized using smooth muscle actin and von Willebrand factor as markers. As shown in FIG. 11, FGF1 increases significantly the vessel density in the scar area. Vascularization is important to supply the muscle with blood. This is true for muscle that is prevented from undergoing apoptosis as well as for newly formed muscle.

### Example 4

### Delivery of p38 Inhibitors and FGF1 via Peptide Nanofibers

Cardiomyocyte Cell Culture

Ventricular cardiomyocytes from 3-day-old Wistar rats (Charles River) were isolated as described (Engel et al., 2005). Neonatal cardiomyocytes were initially cultured for 48 h in the presence of 20 μM cytosine-D-arabinofuranoside (araC; Sigma) and 5% horse serum before stimulation to prevent proliferation of nonmyocytes. Cells were stimulated once with FGF1 (50 ng/mL; R&D Systems). Small molecule inhibitors were added every day.

Myocardial Infarction and Injection of Peptide Nanofibers

Animal experiments were performed in accordance with guidelines of Children's Hospital in Boston and were approved by the Harvard Medical School Standing Committee on Animals. Myocardial infarction (MI) was produced in ˜250 gm male Sprague-Dawley rats (Charles River and Harlan) as described previously (Hsieh et al., 2006). Briefly, rats were anesthetized by pentobarbital and, following tracheal intubation, the hearts were exposed via left thoracotomy. The left coronary artery was identified after pericardiotomy and was ligated by suturing with 6-0 prolene at the location ˜3mm below the left atrial appendix. For the sham operation, suturing was performed without ligation. Peptide nanofibers (peptide sequence AcN-RARADADARARADADA-CNH₂ from Synpep) with BSA (0.1% in PBS) or 400 ng/ml bovine FGF1 (R&D Systems, diluted in 0.1% BSA/PBS) were dissolved in 295 mM sucrose and sonicated to produce 1% solution for injection. Eighty microliters of peptide nanofibers (NF) was injected into the infarcted border zone through three directions immediately after coronary artery ligation. Subsequently, SB203580HCl (Tocris, 2 mg/kg body weight) or saline was injected intraperitoneal, the chest was closed and animals were allowed to recover under a heating pad. Intraperitoneal injection was repeated every 3 days for up to 1 month. For the functional and histological studies, rats were euthanized after 1, 14, or 90 days of surgeries. All of the procedures were blinded and randomized. See, Davis et al., Circulation 2005; 111:442-450, herein incorporated by reference, for further details on nanofiber microenvironments.

Immunofluorescence Staining

Hearts were embedded in tissue-freezing medium (Fisher) without fixation, frozen in 2-methylbutane (cooled in liquid nitrogen), stored at −80° C., and finally sectioned (20 μm; Leica 3050S). Staining was performed as described (Supplementary Table S1) (Engel et al., 2003). Immune complexes were detected with ALEXA 488-, or ALEXA 594-conjugated secondary antibodies (1:400; Molecular Probes). DNA was visualized with DAPI (4,6-diamidino-2-phenylindole, 0.5 μg/mL; Sigma).

Trichrome Stain

Through each heart 7 to 9 sections (1.2 mm interval) from apex to base were subjected to AFOG staining (Poss et al., 2002). Frozen sections were fixed at room temperature (RT) with 10% neutral buffered formalin (10 to 15 min). Sections were permeabilized (0.5% Triton X-100/PBS, 10 min), incubated in preheated Bouins fixative (2.5 hours at 56° C., 1 hour at RT), washed in tap water, incubated in 1% phosphomolybdic acid (5 min), rinsed with destined water, and stained with AFOG staining solution (3 g acid fuchsin, 2 g orange G, 1 g anilin blue dissolved in 200 ml acidified destined water [ph=1.1 HCl], 5 min). Stained sections were rinsed with distilled water, dehydrated with EtOH, cleared in Citrosolv, and mounted. This staining results in a blue coloration of the scar and muscle tissue appears orange/brown. Images were taken for each section to calculate the fibrotic and non-fibrotic areas as well as ventricular and septal wall thickness.

Results

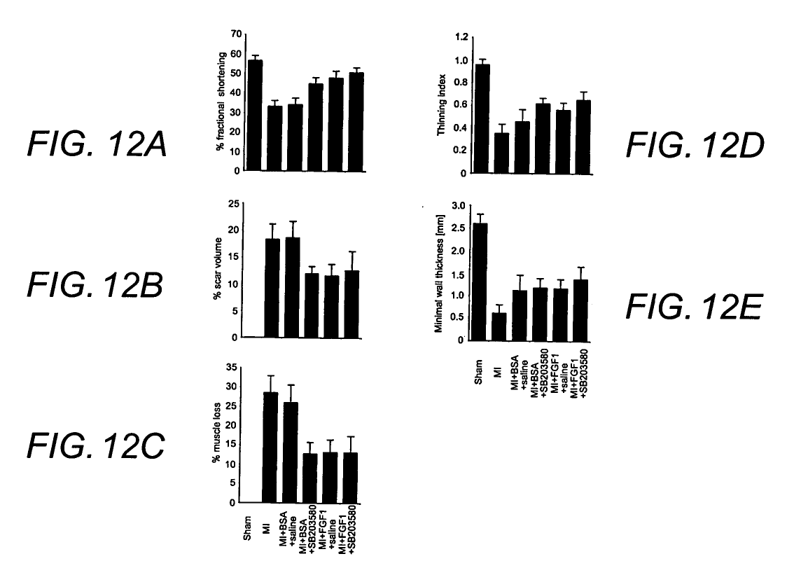

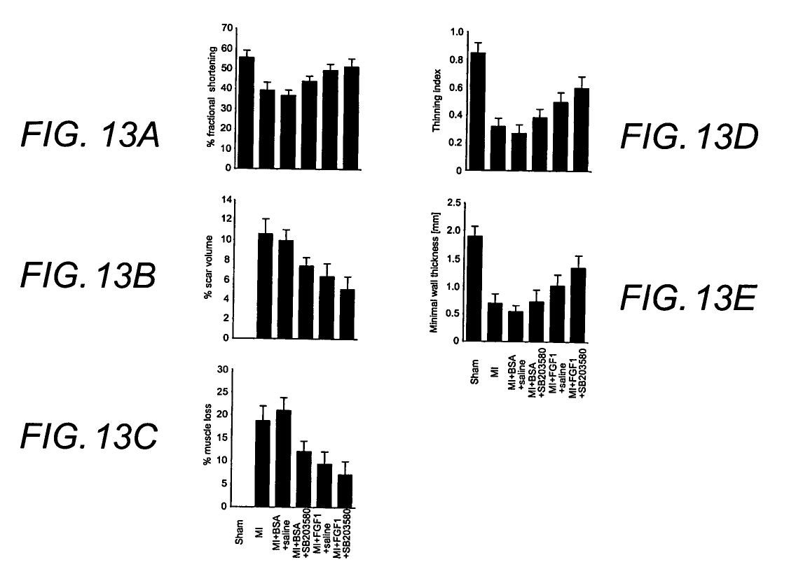

FIGS. 12A-12E provide experimental data for animal sacrificed at 2 weeks. FIG. 12A is a graph illustrating percentage fractional shortening. FIG. 12B is a graph of scar volume. FIG. 12C shows percentage muscle loss. FIG. 12D shows thinning index measurements and FIG. 12E shows wall thickness. Similarly, FIGS. 13A-13E provide experimental data for animal sacrificed at 3 months. FIG. 13A is a graph illustrating percentage fractional shortening. FIG. 13B is a graph of scar volume. FIG. 13C shows percentage muscle loss. FIG. 13D shows thinning index measurements and FIG. 13E shows wall thickness.

Scarring and Thinning

Scar formation was determined as fibrotic area/(fibrotic+non-fibrotic area) based on all sections. The thinning index is a ratio of the amount of wall thinning in the infarct normalized to the thickness of the septum and is calculated by dividing the minimal infarct wall thickness with maximal septal wall thickness (2 weeks: section 1 to 4, 3 month: section 1 to 6 from base).

Echocardiography

Echocardiographic acquisition and analysis were performed as previously described (Lindsey et al., 2002). Left ventricular fractional shortening was calculated as (EDD−ESD)/EDD×100%, where EDD is end-diastolic dimension and ESD is end-systolic dimension.

The invention is also applicable to tissue engineering where cells can be induced to proliferate by treatment with p38 inhibitors or analogs (or such compositions together with growth factors) ex vivo. Following such treatment, the resulting tissue can be used for implantation or transplantation.

While the present invention has been described in terms of specific methods and compositions, it is understood that variations and modifications will occur to those skilled in the art upon consideration of the present invention. Those skilled in the art will appreciate, or be able to ascertain using no more than routine experimentation, further features and advantages of the invention based on the above-described embodiments. Accordingly, the invention is not to be limited by what has been particularly shown and described. All publications and references are herein expressly incorporated by reference in their entirety.

TABLE S1
Names and x-fold changes of clustered genes in FIG. 1A.

| | p38i(1) | FGF(1) | FGF + p38i(1) | p38i(2) | FGF(2) | FGF + p38i(2) |
|---|---|---|---|---|---|---|
| Signal transduction | | | | | | |
| Ephrin B3 | 1.4 | 0.7 | 0.4 | 0.9 | 0.8 | 0.4 |
| RAC2 | 1.6 | 0.7 | 0.4 | 1.1 | 0.6 | 0.3 |
| Rev-ErbA-beta | 1.0 | 0.7 | 0.5 | 1.1 | 0.7 | 0.4 |
| Nr1h3 | 1.1 | 0.7 | 0.5 | 1.4 | 0.6 | 0.4 |
| SPARC related modular calcium | 1.2 | 0.3 | 0.2 | 1.5 | 0.4 | 0.3 |
| binding 2 | | | | | | |
| Ptprc/CD45 | 0.8 | 0.5 | 0.3 | 0.8 | 0.7 | 0.5 |
| Vcam1 | 0.7 | 0.3 | 0.1 | 0.5 | 0.3 | 0.2 |
| Frizzled-related protein 2 | 0.6 | 0.5 | 0.1 | 0.8 | 0.3 | 0.2 |
| Semaphorin 4B | 0.7 | 0.6 | 0.3 | 0.9 | 0.5 | 0.3 |
| Arrestin domain containing 3 | 0.8 | 0.3 | 0.1 | 0.8 | 0.5 | 0.2 |
| Rdc1 | 0.6 | 0.5 | 0.4 | 0.8 | 0.5 | 0.1 |
| Nudt4 | 0.9 | 0.8 | 0.5 | 1.0 | 0.8 | 0.5 |
| IBP6 | 0.9 | 0.6 | 0.5 | 0.9 | 0.9 | 0.5 |
| Ankyrin 2, neuronal | 0.7 | 0.6 | 0.4 | 1.2 | 0.7 | 0.4 |
| Ankyrin repeat domain- | 0.9 | 0.5 | 0.2 | 0.8 | 0.6 | 0.2 |
| containing SOCS box protein 12 | | | | | | |
| Protein-tyrosine phosphatase | 0.7 | 0.5 | 0.2 | 0.9 | 0.6 | 0.2 |
| Igfbp5 | 1.1 | 0.2 | 0.1 | 1.1 | 0.8 | 0.0 |
| Cdc42 effector protein | 0.9 | 0.8 | 0.1 | 1.0 | 0.8 | 0.5 |
| Ghr | 0.8 | 0.9 | 0.5 | 1.1 | 0.8 | 0.5 |
| Fgf16 | 1.0 | 0.8 | 0.5 | 0.8 | 0.8 | 0.4 |
| Egfl3 | 0.7 | 0.4 | 0.3 | 0.5 | 0.5 | 0.3 |
| Osteoglycin | 0.6 | 0.4 | 0.2 | 0.5 | 0.4 | 0.2 |
| CXC chemokine LIX | 0.6 | 0.4 | 0.3 | 0.5 | 0.4 | 0.3 |
| Rbp2 | 0.9 | 0.6 | 0.5 | 0.6 | 0.6 | 0.5 |
| Fcgr2 | 0.9 | 0.5 | 0.4 | 0.5 | 0.6 | 0.3 |
| IGF1 | 0.6 | 0.5 | 0.3 | 0.6 | 0.3 | 0.3 |
| Edg2 | 0.5 | 0.4 | 0.2 | 0.5 | 0.4 | 0.3 |
| Adrenomedullin precursor | 0.5 | 0.4 | 0.4 | 0.7 | 0.6 | 0.3 |
| Lhcgr | 0.6 | 0.4 | 0.5 | 0.6 | 0.8 | 0.4 |
| Ddr2 | 0.6 | 0.5 | 0.4 | 0.7 | 0.8 | 0.5 |
| IGFBP3 | 0.5 | 0.3 | 0.2 | 0.4 | 0.4 | 0.1 |
| Jag1 | 0.6 | 0.4 | 0.2 | 0.4 | 0.6 | 0.2 |
| Multi PDZ domain protein 1 | 0.4 | 0.3 | 0.0 | 0.6 | 0.6 | 0.3 |
| CXCL4 | 0.8 | 0.7 | 0.4 | 0.6 | 0.6 | 0.3 |
| Fzd1 | 0.8 | 0.7 | 0.5 | 0.7 | 0.8 | 0.4 |
| VEGFD | 0.5 | 0.1 | 0.1 | 0.5 | 0.2 | 0.1 |
| RPTPK | 0.7 | 0.4 | 0.4 | 0.6 | 0.4 | 0.4 |
| Figf | 0.7 | 0.3 | 0.3 | 0.6 | 0.3 | 0.3 |
| Epidermal growth factor-like | 0.6 | 0.3 | 0.5 | 0.5 | 0.3 | 0.1 |
| protein, T16 precursor | | | | | | |
| Cxcl12 | 1.4 | 0.5 | 0.4 | 0.8 | 0.3 | 0.2 |
| Tieg | 0.8 | 0.3 | 0.4 | 0.9 | 0.4 | 0.4 |
| Nrtn | 0.6 | 0.4 | 0.3 | 0.9 | 0.6 | 0.5 |
| Ntf3 | 0.7 | 0.2 | 0.1 | 0.7 | 0.3 | 0.2 |
| Mdk | 0.8 | 0.4 | 0.4 | 0.9 | 0.6 | 0.5 |
| Cinc2 | 0.9 | 0.7 | 0.5 | 0.8 | 0.4 | 0.3 |
| Osteoblast specific factor | 0.7 | 0.3 | 0.1 | 0.6 | 0.2 | 0.1 |
| Pleiotrophin | 0.7 | 0.2 | 0.1 | 0.7 | 0.1 | 0.1 |
| Pdgfra | 0.5 | 0.4 | 0.2 | 0.8 | 0.3 | 0.2 |
| Protein tyrosine phosphatase, | 0.7 | 0.5 | 0.5 | 0.8 | 0.5 | 0.1 |
| receptor type, D (Ptprd) | | | | | | |
| FCEG | 1.1 | 0.7 | 0.4 | 0.8 | 0.5 | 0.5 |
| Semaphorin 6D-1 | 1.0 | 0.5 | 0.3 | 0.8 | 0.3 | 0.2 |
| Emr1 | 1.1 | 0.6 | 0.3 | 0.9 | 0.4 | 0.4 |
| Fcgr3 | 1.0 | 0.5 | 0.3 | 0.9 | 0.6 | 0.4 |
| Rho-related BTB domain | 0.9 | 0.5 | 0.4 | 1.0 | 0.6 | 0.4 |
| containing 3 | | | | | | |
| Gab1 | 1.0 | 0.6 | 0.5 | 0.9 | 0.7 | 0.5 |
| PAPIN | 0.9 | 0.5 | 0.4 | 0.8 | 0.7 | 0.3 |
| Fbln5 | 0.3 | 0.4 | 0.3 | 0.4 | 0.6 | 0.2 |
| Cish | 0.3 | 0.4 | 0.3 | 0.3 | 0.5 | 0.2 |
| AGTR1 | 0.4 | 0.4 | 0.3 | 0.3 | 0.5 | 0.2 |
| Vegfc | 0.5 | 0.4 | 0.5 | 0.7 | 0.6 | 0.5 |
| Agtr1a | 0.6 | 0.6 | 0.5 | 0.4 | 0.4 | 0.3 |
| Bmp3 | 0.3 | 0.5 | 0.5 | 0.2 | 0.6 | 0.3 |
| Growth hormone receptor | 0.5 | 0.7 | 0.5 | 0.8 | 0.8 | 0.5 |
| Adrenergic, alpha 1B-, receptor | 0.6 | 0.8 | 0.4 | 0.5 | 0.7 | 0.3 |
| Rgpr | 0.6 | 0.6 | 0.4 | 0.6 | 0.9 | 0.5 |
| Cish3 | 0.6 | 0.8 | 0.5 | 0.6 | 0.9 | 0.4 |
| Connexin 40 | 0.5 | 0.8 | 0.4 | 0.5 | 0.8 | 0.2 |
| Cish2 | 0.4 | 0.6 | 0.4 | 0.4 | 0.8 | 0.3 |
| CISH | 0.3 | 0.4 | 0.3 | 0.3 | 0.5 | 0.2 |
| Serine-threonine specific protein | 0.6 | 0.7 | 0.5 | 0.4 | 0.7 | 0.5 |
| phosphatase, GL subunit | | | | | | |
| Wisp2 | 0.3 | 0.8 | 0.4 | 0.3 | 0.8 | 0.2 |
| Seta | 0.7 | 1.0 | 0.3 | 0.7 | 1.0 | 0.4 |
| MCP-3 | 0.7 | 1.3 | 0.4 | 0.6 | 0.7 | 0.4 |
| Epha3 | 0.4 | 0.5 | 0.2 | 0.5 | 0.5 | 0.1 |
| Tgfbr2 | 0.5 | 0.5 | 0.4 | 0.9 | 0.9 | 0.3 |
| RhoGAP | 0.7 | 0.8 | 0.5 | 0.8 | 0.8 | 0.4 |
| Retinoic acid receptor, gamma | 0.8 | 0.6 | 0.3 | 0.7 | 1.0 | 0.5 |
| Nr2f2/COUP-TFII | 0.8 | 0.8 | 0.3 | 0.8 | 0.8 | 0.4 |
| Connexin 43 | 0.9 | 0.9 | 0.3 | 0.7 | 0.8 | 0.5 |
| Casein kinase I delta | 1.0 | 1.3 | 0.5 | 0.8 | 0.9 | 0.5 |
| Protease inhibitor 7 | 0.5 | 1.3 | 0.5 | 0.7 | 0.8 | 0.4 |
| Hepatocyte F2alpha receptor | 0.5 | 0.9 | 0.4 | 0.6 | 1.0 | 0.4 |
| PTP-RL9, receptor-type protein | 0.6 | 1.1 | 0.4 | 0.2 | 0.8 | 0.0 |
| tyrosine phosphatase | | | | | | |
| Integrin, alpha 11 | 0.1 | 1.7 | 0.3 | 0.3 | 1.2 | 0.3 |
| Soluble fibroblast growth factor | 1.1 | 0.9 | 2.2 | 0.5 | 0.8 | 2.1 |
| receptor IIIb (sKGF-R gene) | | | | | | |
| Cacalmodulin-dependent protein | 0.8 | 0.9 | 2.0 | 0.8 | 1.3 | 2.0 |
| kinase phosphatase | | | | | | |
| II1 rap | 1.7 | 2.1 | 4.0 | 5.4 | 6.4 | 12.2 |
| CRE-BPA, delta chain | 1.6 | 0.9 | 2.3 | 6.1 | 10.9 | 21.6 |
| MCIP1 | 2.0 | 3.0 | 3.9 | 2.0 | 3.3 | 4.9 |
| Rho family GTPase 1 | 4.0 | 4.1 | 7.7 | 3.9 | 3.9 | 6.7 |
| Efna5 | 0.8 | 2.1 | 2.0 | 6.5 | 5.9 | 8.6 |
| GOA1B | 2.0 | 2.9 | 3.4 | 2.5 | 3.0 | 3.2 |
| Flt4/VEGFR3 | 1.7 | 1.8 | 3.7 | 2.8 | 2.5 | 4.3 |
| ROC2 | 1.1 | 1.6 | 2.9 | 3.5 | 2.4 | 4.5 |
| TRAF4 associated factor 1 | 2.1 | 1.9 | 3.4 | 2.2 | 1.9 | 3.3 |
| PSCD3 | 2.1 | 10.1 | 5.9 | 2.0 | 2.1 | 3.2 |
| Snf1lk | 1.2 | 3.5 | 3.0 | 2.0 | 3.9 | 3.5 |
| Vegfr3 | 0.9 | 0.8 | 2.0 | 1.9 | 1.7 | 3.6 |
| Nuclear receptor co-activator | 1.5 | 1.4 | 2.7 | 1.4 | 1.2 | 2.1 |
| NRIF3 | | | | | | |
| Tgf beta 2 | 1.0 | 1.4 | 3.5 | 1.3 | 1.5 | 3.3 |
| EphA2 | 1.5 | 2.2 | 4.2 | 1.0 | 1.7 | 2.2 |
| Catenin, alpha-1 | 1.2 | 1.6 | 2.5 | 1.0 | 1.5 | 2.0 |
| Lgals1 | 1.3 | 1.8 | 2.3 | 1.2 | 1.7 | 2.7 |
| PRKAR2 | 1.4 | 1.6 | 2.9 | 1.3 | 1.2 | 2.9 |
| GDF15 | 1.1 | 1.6 | 3.2 | 2.3 | 2.6 | 7.8 |
| BMP6 precursor | 1.4 | 1.4 | 2.4 | 1.3 | 1.7 | 3.2 |
| (BEM)-3 | 1.3 | 1.4 | 2.1 | 1.5 | 1.6 | 2.3 |
| Cgef2 | 1.0 | 0.8 | 2.5 | 4.3 | 7.9 | 24.6 |
| Pparg | 2.4 | 1.3 | 2.7 | 11.0 | 3.3 | 13.1 |
| Rac GTPase-activating protein 1 | 2.0 | 1.3 | 3.2 | 2.7 | 1.9 | 3.3 |
| Shc SH2-domain binding protein 1 | 2.7 | 1.6 | 4.2 | 1.8 | 1.1 | 2.4 |
| Integrin, alpha 6 | 1.1 | 2.4 | 2.5 | 0.8 | 2.5 | 3.3 |
| Tspan-2 | 0.9 | 2.4 | 2.8 | 0.9 | 1.8 | 2.3 |
| Ctgf | 1.0 | 1.9 | 2.4 | 0.9 | 1.6 | 2.0 |
| Antisense basic fibroblast growth | 0.2 | 1.3 | 2.3 | 1.6 | 3.4 | 4.3 |
| factor, Nudt6 | | | | | | |
| Lgals9 | 0.8 | 4.3 | 8.8 | 1.0 | 3.1 | 4.7 |
| BMP2 | 1.0 | 6.0 | 6.1 | 1.3 | 3.4 | 3.5 |
| Mena/RNB6 | 2.9 | 11.0 | 17.5 | 1.2 | 2.3 | 5.9 |
| SFRP1 | 1.5 | 4.9 | 6.0 | 1.3 | 2.2 | 3.2 |
| Dok-5 | 1.1 | 2.7 | 2.1 | 1.6 | 2.6 | 4.7 |
| LDLR | 1.5 | 4.0 | 5.4 | 1.3 | 2.7 | 4.2 |
| Pdgfc | 1.7 | 3.4 | 2.9 | 0.8 | 1.2 | 2.0 |
| Semaphorin 4G | 0.9 | 1.0 | 2.2 | 1.7 | 4.1 | 3.5 |
| Melusin | 1.4 | 3.8 | 2.6 | 1.3 | 3.0 | 4.5 |
| Transferrin receptor | 1.3 | 2.9 | 3.4 | 0.9 | 1.6 | 2.1 |
| IL12a | 1.4 | 2.9 | 2.2 | 1.0 | 2.8 | 5.4 |
| Cadherin 13 | 1.0 | 1.5 | 2.0 | 1.3 | 1.9 | 2.3 |
| Plasminogen activator inhibitor 1 | 0.8 | 5.3 | 4.4 | 0.7 | 8.8 | 3.2 |
| Unc5h2 | 0.7 | 1.7 | 4.7 | 0.8 | 1.3 | 2.7 |
| Retinoic acid inducible protein 3 | 1.1 | 1.5 | 3.2 | 0.8 | 1.3 | 2.0 |
| VEGF receptor-2FLK-1 | 0.8 | 1.8 | 2.3 | 0.6 | 1.6 | 3.5 |
| Tspan-6 | 0.7 | 2.2 | 3.2 | 0.8 | 1.7 | 3.1 |
| Rgs3 | 0.8 | 1.3 | 2.3 | 1.2 | 1.9 | 2.5 |
| DUSP6 | 0.9 | 2.8 | 11.2 | 0.8 | 4.4 | 4.6 |
| Lgals5 | 0.7 | 2.9 | 5.7 | 1.2 | 2.9 | 5.2 |
| MKK6 | 7.7 | 0.2 | 3.7 | 5.6 | 0.1 | 2.9 |
| Fetal genes/cytoskeleton | | | | | | |
| Aif1 | 0.8 | 0.9 | 0.5 | 0.5 | 0.4 | 0.1 |
| FHOS2 | 0.9 | 0.7 | 0.4 | 1.0 | 0.8 | 0.5 |
| Troponin I, slow isoform | 1.1 | 0.6 | 0.5 | 0.9 | 0.6 | 0.5 |
| Cfl1 | 1.0 | 0.6 | 0.5 | 0.9 | 0.6 | 0.5 |
| Mtss1 | 0.8 | 0.5 | 0.3 | 0.9 | 0.5 | 0.3 |
| Enigma | 1.3 | 2.3 | 4.8 | 1.4 | 2.3 | 4.9 |
| Actinin | 1.3 | 1.8 | 3.1 | 1.0 | 1.5 | 2.5 |
| Actn1 | 1.4 | 1.9 | 4.0 | 1.3 | 1.9 | 3.1 |
| Desmin | 1.2 | 2.2 | 3.3 | 1.0 | 1.8 | 3.0 |
| Calponin 1 | 1.3 | 6.4 | 13.3 | 1.4 | 4.9 | 12.3 |
| MLC 2a | 1.2 | 2.7 | 3.8 | 0.9 | 2.1 | 3.2 |
| Moesin | 0.9 | 2.2 | 3.8 | 0.9 | 2.4 | 3.7 |
| Smooth muscle 22 protein | 0.9 | 1.2 | 2.9 | 1.0 | 1.4 | 2.8 |
| Nppa | 2.3 | 3.1 | 4.4 | 1.9 | 3.5 | 4.3 |
| NppB | 4.5 | 10.4 | 13.6 | 4.4 | 11.6 | 14.9 |
| Acta1 | 1.7 | 4.8 | 4.3 | 1.2 | 6.3 | 7.6 |
| M-protein | 1.3 | 8.7 | 8.0 | 1.6 | 11.0 | 10.4 |
| MLC3, alkali | 0.5 | 13.9 | 3.2 | 1.1 | 23.2 | 11.6 |
| MLC1, atrial isoform | 1.2 | 3.3 | 2.0 | 0.8 | 2.8 | 2.8 |
| Myopodin | 0.8 | 3.7 | 3.5 | 0.8 | 3.2 | 2.2 |
| G2/M, cytokinesis | | | | | | |
| Anillin | 2.2 | 3.3 | 5.3 | 2.0 | 1.9 | 3.5 |
| Tubulin, beta 2 | 1.5 | 1.3 | 2.0 | 1.5 | 1.2 | 2.0 |
| Diaphanous homolog 3 | 1.4 | 2.5 | 4.7 | 1.9 | 1.9 | 2.8 |
| MAD2-LIKE 1 | 1.3 | 1.5 | 2.2 | 2.1 | 2.8 | 4.7 |
| Cdc25B | 1.3 | 1.6 | 2.9 | 1.5 | 1.3 | 2.2 |
| Tubulin alpha-4 | 1.4 | 2.7 | 3.1 | 1.4 | 2.6 | 4.0 |
| Mapt | 5.9 | 1.7 | 3.6 | 3.9 | 1.9 | 2.5 |
| ORC6 | 4.2 | 1.7 | 3.3 | 1.9 | 0.5 | 2.6 |
| Cyclin B1 | 1.6 | 1.2 | 2.6 | 1.6 | 1.0 | 3.0 |
| Cdca3 | 2.3 | 1.3 | 3.2 | 1.9 | 1.4 | 2.3 |
| Bub1 | 2.4 | 1.4 | 2.5 | 1.4 | 1.2 | 2.8 |
| Tubulin beta class I | 1.9 | 2.4 | 4.8 | 1.6 | 1.9 | 3.5 |
| PTTG1 | 2.1 | 1.4 | 3.1 | 1.9 | 1.4 | 2.7 |
| Stathmin | 2.7 | 1.2 | 2.5 | 2.0 | 1.6 | 2.3 |
| NuSAP | 2.4 | 1.5 | 2.5 | 2.2 | 1.4 | 2.4 |
| MPP1 | 2.0 | 1.4 | 2.8 | 3.3 | 2.0 | 4.2 |
| Aurora B | 2.6 | 1.5 | 3.8 | 3.1 | 1.9 | 3.5 |
| Topoisomerase (DNA) II alpha | 2.8 | 1.8 | 3.1 | 2.9 | 1.7 | 4.0 |
| CKS2 | 2.4 | 1.7 | 2.7 | 1.7 | 1.4 | 2.1 |
| Cdc2a | 2.6 | 1.7 | 3.4 | 2.1 | 1.8 | 2.9 |
| Kinetochore-associated protein 1 | 2.1 | 1.9 | 3.0 | 2.7 | 2.1 | 3.6 |
| MPS1 | 2.1 | 0.9 | 3.3 | 7.5 | 6.6 | 9.6 |
| Kinesin family member 22 | 1.8 | 1.6 | 3.1 | 2.0 | 1.7 | 2.4 |
| MCAK | 2.5 | 1.8 | 3.9 | 2.0 | 1.7 | 3.1 |
| Cdc20 | 2.1 | 1.9 | 4.0 | 1.8 | 1.7 | 2.7 |
| CENP-E | 1.8 | 1.3 | 2.9 | 1.6 | 1.7 | 2.2 |
| Septin 6 | 1.6 | 1.9 | 2.8 | 2.3 | 1.9 | 4.3 |
| Spag5 | 1.7 | 1.4 | 2.9 | 1.7 | 1.9 | 2.6 |
| Prc1 | 2.2 | 2.4 | 4.8 | 2.1 | 1.9 | 3.7 |
| Ect2 | 2.5 | 1.9 | 4.6 | 2.4 | 2.5 | 4.1 |
| Cyclin B | 2.0 | 1.9 | 3.4 | 1.8 | 1.8 | 3.0 |
| Proliferation/regeneration | | | | | | |
| Testin | 0.6 | 0.6 | 0.3 | 0.6 | 0.7 | 0.5 |
| P57 | 0.3 | 0.2 | 0.1 | 0.3 | 0.3 | 0.1 |
| BOC | 0.8 | 0.5 | 0.2 | 0.8 | 0.6 | 0.2 |
| G0S2-like protein | 1.0 | 0.5 | 0.2 | 1.0 | 0.6 | 0.3 |
| Dri42 | 0.7 | 0.5 | 0.3 | 0.8 | 0.3 | 0.2 |
| Glypican (GPC)-3 | 0.8 | 0.4 | 0.3 | 0.8 | 0.5 | 0.4 |
| RNA binding motif protein 5 | 0.9 | 0.6 | 0.5 | 0.9 | 0.5 | 0.5 |
| Prg-1 | 0.7 | 0.2 | 0.4 | 0.8 | 0.3 | 0.2 |
| MDP77 | 0.4 | 1.5 | 0.2 | 0.3 | 0.8 | 0.3 |
| Ptpla | 1.2 | 3.3 | 3.2 | 0.8 | 2.2 | 2.8 |
| Mustang | 0.9 | 4.8 | 9.5 | 1.5 | 5.2 | 9.6 |
| Thymidine kinase | 2.0 | 1.5 | 3.3 | 1.8 | 1.9 | 3.3 |
| CDKN3/KAP/CDI1 | 1.8 | 1.7 | 4.2 | 1.8 | 2.0 | 4.1 |
| Ki67 | 1.4 | 1.7 | 3.4 | 1.5 | 1.5 | 2.7 |
| STK38 | 2.3 | 3.9 | 6.1 | 1.4 | 2.0 | 4.4 |
| Growth response protein (CL-6) | 1.8 | 2.5 | 3.9 | 1.5 | 1.8 | 3.1 |
| Tmeff1 | 1.4 | 2.1 | 3.9 | 1.3 | 2.4 | 2.9 |
| Tnfrsf12a | 1.7 | 3.7 | 7.1 | 1.7 | 3.3 | 6.0 |
| Cyclin D1 | 1.9 | 4.2 | 6.1 | 1.8 | 3.3 | 6.0 |
| Cyclin A2 | 2.7 | 1.8 | 6.0 | 2.6 | 1.1 | 3.6 |
| RAD51 | 2.2 | 1.5 | 3.3 | 1.7 | 1.0 | 2.1 |
| GADD45a | 2.5 | 1.8 | 3.1 | 2.3 | 1.3 | 2.9 |
| STk18 | 2.3 | 1.3 | 2.6 | 2.4 | 1.9 | 3.3 |
| GADD45 gamma | 2.6 | 3.0 | 4.5 | 2.5 | 2.9 | 5.2 |
| Geminin | 2.5 | 2.3 | 4.7 | 15.1 | 10.7 | 22.3 |
| Fancd2 | 3.4 | 0.7 | 4.0 | 1.7 | 1.1 | 2.1 |
| Transcriptional control | | | | | | |
| Id3a | 0.9 | 2.4 | 2.6 | 0.7 | 1.9 | 2.5 |
| ETV5/ERM/PEA3 | 1.2 | 20.4 | 30.3 | 0.3 | 11.2 | 27.1 |
| Id1 | 0.9 | 2.7 | 4.9 | 1.0 | 2.3 | 3.5 |
| FHL1 | 1.6 | 7.3 | 7.6 | 1.0 | 5.1 | 8.1 |
| TBX2 | 1.0 | 1.8 | 2.1 | 1.3 | 2.0 | 2.4 |
| CSRP1 | 1.0 | 1.9 | 4.4 | 1.1 | 1.8 | 3.3 |
| Csrp3/MLP | 2.5 | 2.7 | 2.6 | 2.5 | 4.2 | 3.8 |
| CREB5 | 3.0 | 1.3 | 3.9 | 1.6 | 1.8 | 3.3 |
| Polyamine-modulated factor 1 | 1.9 | 1.5 | 3.9 | 1.2 | 1.4 | 2.5 |
| Sox11 | 1.2 | 0.9 | 2.6 | 1.1 | 1.3 | 2.2 |
| Nfix | 0.5 | 0.3 | 0.5 | 0.7 | 0.7 | 0.5 |
| Osterix | 0.8 | 0.6 | 0.3 | 0.8 | 0.6 | 0.5 |
| Sox4 | 0.9 | 0.7 | 0.3 | 0.7 | 0.6 | 0.4 |
| SHARP-1/dec2/Bhlhb3 | 0.7 | 0.2 | 0.1 | 0.8 | 0.3 | 0.1 |
| Sponf | 0.7 | 0.2 | 0.1 | 0.8 | 0.3 | 0.1 |
| Maf-2 | 0.9 | 0.5 | 0.3 | 0.6 | 0.5 | 0.5 |
| IRX4 | 0.7 | 0.7 | 0.3 | 1.2 | 0.9 | 0.4 |
| MURF1 | 1.4 | 0.6 | 0.5 | 1.0 | 0.5 | 0.5 |
| Kruppel-like factor 4 | 0.7 | 1.1 | 0.5 | 0.7 | 1.1 | 0.5 |
| Kruppel-like factor 2 | 0.5 | 0.7 | 0.1 | 0.6 | 0.9 | 0.5 |
| MURF | 0.6 | 0.8 | 0.3 | 0.6 | 0.9 | 0.4 |
| Pem | 0.8 | 0.9 | 0.5 | 0.7 | 0.7 | 0.5 |
| Lisch7 | 0.3 | 0.6 | 0.3 | 0.4 | 0.8 | 0.5 |
| Apoptosis | | | | | | |
| PEA-15 | 1.3 | 2.5 | 3.3 | 1.2 | 2.1 | 2.6 |
| Sh3kbp1 | 1.0 | 1.1 | 0.4 | 0.6 | 0.9 | 0.4 |
| Bcl2a1 | 0.6 | 0.4 | 0.5 | 0.9 | 0.6 | 0.5 |
| BimL | 0.5 | 0.3 | 0.3 | 1.0 | 0.7 | 0.5 |
| BCl2l11/BIM | 0.7 | 0.5 | 0.5 | 0.9 | 1.1 | 0.5 |
| Apoptosis protein MA-3 | 0.9 | 0.8 | 0.5 | 0.8 | 0.7 | 0.4 |
| CABC1 | 0.7 | 0.6 | 0.2 | 0.7 | 0.5 | 0.2 |
| Lot1 | 0.7 | 0.3 | 0.1 | 0.6 | 0.4 | 0.2 |
| DAPK1 | 1.1 | 0.6 | 0.4 | 1.2 | 0.5 | 0.3 |
| Pdcd4 | 1.1 | 0.8 | 0.5 | 0.9 | 0.7 | 0.4 |

TABLE S2
Induction of DNA synthesis in neonatal cardiomyocytes.

| | | | BrdU labeling period: | | | | |
|---|---|---|---|---|---|---|---|
| | | | 24 to 48 hours | | 48 to 72 hours | | 24 to 72 hours |
| | | | Harvested after: | | | | |
| | | | 48 hours | | 72 hours | | 72 hours |
| | concentration (ng/ml) | | % of BrdU positive neonatal cardiomyoctes | | | | |
| Stimulus: | low | high | low | high | low | high | optimal |
| BMP6 | 40 | 200 | 5.3 ± 0.6 | 5.8 ± 1.4 | 3.5 ± 0.6 | 4.0 ± 0.7 | |
| BMP7 | 20 | 100 | 3.1 ± 0.6 | 4.8 ± 1.8 | 6.2 ± 0.3 | 7.2 ± 0.5 | |
| Chordin | 100 | 500 | 3.1 ± 1.1 | 2 ± 0.6 | 2.1 ± 0.9 | 2.4 ± 0.6 | |
| CT-1 | 20 | 100 | 8.5 ± 1.5 | 11.1 ± 0.7 | 11.5 ± 2.1 | 8.9 ± 1.5 | 11.2 ± 1.1 |
| EGF | 100 | 500 | 5.4 ± 0.8 | 6.1 ± 0.2 | 6.7 ± 0.8 | 6.3 ± 0.8 | |
| FGF1 | 50 | 250 | 39.5 ± 2.4 | 31.8 ± 1.6 | 32.8 ± 2.2 | 26.3 ± 1.5 | 61.8 ± 2.1 |
| FGF2 | 50 | 250 | 12.9 ± 1.6 | 17.1 ± 1.6 | 9.3 ± 0.6 | 11.9 ± 1.0 | 22.2 ± 1.8 |
| FGF4 | 50 | 250 | 22.1 ± 2.4 | 16.5 ± 1.8 | 20.0 ± 0.3 | 15.7 ± 0.9 | 46.5 ± 3.9 |
| FGF5 | 50 | 250 | 5.5 ± 0.7 | 6.4 ± 0.7 | 4.7 ± 1.2 | 5.0 ± 0.7 | |
| FGF6 | 50 | 250 | 30.5 ± 2.2 | 22.9 ± 2.0 | 27.3 ± 1.7 | 19.1 ± 1.5 | 47.3 ± 3.3 |
| FGF7 | 20 | 100 | 4.0 ± 1.1 | 3.9 ± 1.0 | 4.3 ± 2.0 | 3.9 ± 0.5 | |
| FGF8b | 50 | 250 | 9.4 ± 1.3 | 16.0 ± 1.3 | 7.7 ± 2.2 | 21.5 ± 3.0 | 29.2 ± 1.9 |
| FGF8c | 50 | 250 | 3.7 ± 1.0 | 3.9 ± 0.2 | 5.1 ± 1.0 | 5.0 ± 1.6 | |
| FGF9 | 50 | 250 | 13.1 ± 2.3 | 16.5 ± 2.3 | 19.9 ± 2.0 | 23.1 ± 1.1 | 37.1 ± 2.4 |
| FGF10 | 50 | 250 | 3.9 ± 0.8 | 4.5 ± 0.6 | 3.0 ± 0.7 | 2.3 ± 0.5 | |
| FGF17 | 50 | 250 | 5.7 ± 1.4 | 16.3 ± 1.3 | 5.2 ± 2.8 | 19.5 ± 1.7 | 31.1 ± 1.7 |
| FGF18 | 50 | 250 | 6.0 ± 0.5 | 5.3 ± 0.9 | 3.5 ± 0.3 | 4.2 ± 0.7 | |
| FGF19 | 50 | 250 | 4.9 ± 1.6 | 4.1 ± 1.1 | 3.0 ± 1.1 | 3.1 ± 0.6 | |
| FS300 | 50 | 250 | 3.3 ± 0.4 | 3.5 ± 0.8 | 2.0 ± 0.5 | 1.5 ± 0.4 | |
| GDF5 | 100 | 500 | 5.5 ± 1.4 | 5.2 ± 0.9 | 3.9 ± 0.8 | 5.5 ± 0.9 | |
| GDF6 | 100 | 500 | 2.3 ± 0.5 | 2.8 ± 0.9 | 2.2 ± 0.7 | 2.1 ± 0.8 | |
| GDF7 | 20 | 100 | 3.5 ± 0.9 | 3.8 ± 0.7 | 4.6 ± 1.5 | 4.7 ± 0.6 | |
| GDF8 | 20 | 100 | 5.9 ± 0.7 | 5.1 ± 0.3 | 3.9 ± 1.7 | 3.5 ± 1.7 | |
| HGF | 50 | 250 | 5.8 ± 1.1 | 6.3 ± 0.7 | 5.7 ± 0.8 | 4.7 ± 0.4 | |
| IFNγ | 100 | 500 | 1.3 ± 0.1 | 1.9 ± 0.3 | 1.2 ± 0.4 | 2.3 ± 0.4 | |
| IGF1 | 100 | 500 | 6.3 ± 1.0 | 6.1 ± 1.5 | 4.5 ± 1.5 | 5.1 ± 1.3 | |
| IGF2 | 100 | 500 | 2.7 ± 0.9 | 4.9 ± 1.2 | 1.9 ± 0.3 | 2.9 ± 0.3 | |
| IL-1β | 20 | 100 | 10.1 ± 1.1 | 17.2 ± 1.9 | 10.7 ± 2.2 | 18.7 ± 2.4 | 41.3 ± 3.2 |
| IL3 | 20 | 100 | 1.9 ± 0.1 | 2.3 ± 0.4 | 2.7 ± 0.6 | 1.8 ± 0.6 | |
| IL6 | 10 | 50 | 4.6 ± 0.3 | 3.1 ± 0.6 | 5.7 ± 1.3 | 4.2 ± 0.8 | |
| IL10 | 10 | 50 | 7.4 ± 1.0 | 4.9 ± 0.5 | 4.1 ± 0.3 | 3.6 ± 1.1 | |
| IL11 | 10 | 50 | 7.0 ± 1.2 | 4.1 ± 1.9 | 3.6 ± 0.5 | 4.4 ± 1.1 | |
| Midkine | 100 | 500 | 1.5 ± 0.4 | 2.0 ± 0.5 | 2.3 ± 0.3 | 2.9 ± 0.5 | |
| Noggin | 100 | 500 | 2.3 ± 0.6 | 2.4 ± 0.2 | 2.1 ± 0.7 | 1.6 ± 0.8 | |
| NRG-1-β1 | 50 | 250 | 26.3 ± 1.9 | 22.2 ± 2.0 | 26.1 ± 2.5 | 10.2 ± 2.6 | 46.4 ± 1.5 |
| NT3 | 10 | 50 | 6.1 ± 0.4 | 5.4 ± 0.9 | 3.2 ± 0.8 | 6.1 ± 0.8 | |
| NT4 | 10 | 50 | 2.6 ± 0.5 | 2.4 ± 0.5 | 3.7 ± 0.6 | 3.7 ± 0.5 | |
| Pleiotrophin | 100 | 500 | 4.5 ± 1.2 | 6.5 ± 1.0 | 3.7 ± 0.5 | 3.6 ± 0.5 | |
| TGFα | 100 | 500 | 3.8 ± 1.1 | 4.5 ± 1.0 | 3.9 ± 0.8 | 4.5 ± 1.1 | |
| TGFβ1 | 4 | 20 | 11.9 ± 0.9 | 20.5 ± 1.3 | 13.5 ± 1.8 | 18.7 ± 1.3 | 17.4 ± 2.2 |
| TGFβ2 | 4 | 20 | 8.7 ± 0.8 | 12 ± 1.1 | 11.1 ± 1.4 | 12.7 ± 1.5 | |
| TGFβ3 | 4 | 20 | 7.8 ± 1.2 | 6.1 ± 1.0 | 8.6 ± 1.4 | 5.8 ± 0.9 | |
| TNFα | 20 | 100 | 2.7 ± 0.9 | 2.8 ± 0.5 | 3.4 ± 0.4 | 3.3 ± 0.8 | |
| PE | 20 μM | 100 μM | 7.9 ± 1.3 | 11.4 ± 1.7 | 12.5 ± 1.5 | 10.4 ± 1.2 | 23.7 ± 3.1 |
| FBS | 10% | 20% | 18.5 ± 1.5 | 22.5 ± 3.2 | 17.7 ± 2.0 | 20.3 ± 2.1 | 35.5 ± 2.4 |
| BSA | 0.1% | | 2.0 ± 0.3 | | 1.6 ± 0.3 | | 2.1 ± 0.3 |
| DMSO | 0.2% | | 3.2 ± 0.7 | | 2.4 ± 0.2 | | 2.7 ± 0.8 |

TABLE S3
Information for immunofluorescence staining and Western blotting

| Antibody | Dilution | Source | Incubation |
|---|---|---|---|
| Immunofluorescence | | | |
| Staining: | | | |
| Tropomyosin | 1:100 | DSHB, J. J.-C. Lin | RT, 1 h |
| Troponi T | 1:100 | DSHB, J. J.-C. Lin | RT, 1 h |
| H3P (mouse/rabbit) | 1:200/1:100 | Upstate | RT, 1 h |
| BrdU | 1:100 | Abeam | RT, 1 h |
| Aurora B | 1:250 | Transduction Laboratories | RT, 1 h |
| Caveolin 3 | 1:100 | Transduction Laboratories | RT, 1 h |
| p27 | 1:50 | Transduction Laboratories | RT, 1 h |
| Troponin I | 1:50 | Santa Cruz | RT, 1 h |
| MEF2 | 1:200 | Santa Cruz | RT, 1 h |
| GATA | 1:100 | Santa Cruz | RT, 1 h |
| Survivin | 1:50 | Santa Cruz | RT, 1 h |
| Cyclin A | 1:50 | Santa Cruz | RT, 1 h |
| Cdc2 | 1:50 | Santa Cruz | RT, 1 h |
| Flag(M2) | 1:500 | Santa Cruz | RT, 1 h |
| Ki67 | 1:50 | AbCam | RT, 1 h |
| PRb807/811 | 1:100 | Cell Signaling | RT, 1 h |
| Nkx2.5 | 1:500 | Kasahara et al., 1998 | 4° C. over night |
| Western Blotting: | | | |
| Phospho ATF-2 | 1:1000 | Cell Signaling | 4° C. over night |
| Phospho Akt (Ser473) | 1:1000 | Cell Signaling | 4° C. over night |
| Phospho Akt (Thr308) | 1:1000 | Cell Signaling | 4° C. over night |
| p38 | 1:1000 | Cell Signaling | 4° C. over night |
| Actin (Ab1) | 1:1000 | Oncogene | 4° C. over night |
| p38α | 1:1000 | Cell Signaling | 4° C. over night |
| p38β | 1:1000 | gift from Dr. J. Han, Scripps | 4° C. over night |
| | | Research Institute | |
| p38γ | 1:1000 | gift from Dr. J. Han, Scripps | 4° C. over night |
| | | Research Institute | |
| phospho-p38 | 1:1000 | Cell Signaling | 4° C. over night |
| MAPKAPK2 | 1:1000 | Cell Signaling | 4° C. over night |
| phospho-ERK | 1:2000 | Cell Signaling | 4° C. over night |

TABLE S4

Information for RT-PCR

| | | | |
|---|---|---|---|
| Forward | Reverse | Annealing | Size |

| | | | | |
|---|---|---|---|---|
| Name | primer* | primer* | (° C.) | (bp) |

| | | | | |
|---|---|---|---|---|
| Seta | 5′ gcgCAATAAA | 5′ gcgTTTGATG | 56 | 772 |
| | CGAGGAGAGCGAC | ACAGGAGCGGATG | | |
| | A 3′ | G 3′ | | |
| Dusp6 | 5′ gcgCATCTCT | 5′ gcgTCTCTCC | 56 | 328 |
| | CCCAACTTCAACT | CTCCGTAATAACC | | |
| | T 3′ | A 3′ | | |
| Desmin | 5′ gcgAGGAGAT | 5′ gcgTGTGAGA | 56 | 555 |
| | GATGGAATACCGA | GGAGAAAAGCGAC | | |
| | C 3′ | T 3′ | | |
| ANP | 5′ gcgTGAGCGA | 5′ gcgTCAATCC | 58 | 220 |
| | GCAGACCGATGAA | TACCCCCGAAGCA | | |
| | G 3′ | G 3′ | | |
| BNP | 5′ gcgAGCCAGT | 5′ gcgTAAAACA | 56 | 269 |
| | CTCCAGAACAATC | ACCTCAGCCCGTC | | |
| | C 3′ | A 3′ | | |
| Top2a | 5′ gcgCTGAGTT | 5′ gcGAAGACGA | 54 | 360 |
| | TGAGAAGGCGATT | CAATGCCCACGAG | | |
| | T 3′ | 3′ | | |
| Cdc2a | 5′ gcgAAAATAG | 5′ gcgCGGGAGT | 53 | 350 |
| | AGAAAATCGGAGA | GACAAAACACAAT | | |
| | A 3 | C 3′ | | |
| Ect2 | 5′ AGCCCTTGCC | 5′ CCCGTTGTCC | 53 | 564 |
| | GTTCTCCTGCC | TTCTTCTTCTA | | |
| | 3′ | 3′ | | |
| Cyclin B | 5′ gcgTAAAGTC | 5′ gcGGAGAGGG | 53 | 204 |
| | AGCGAACAGTCAA | AGTATCAACCAAA | | |
| | G 3′ | 3′ | | |
| MUSTANG | 5′ gcgTGCTGCC | 5′ gcgACACACA | 56 | 556 |
| | AGAGAGTTACCAA | TCATTCCCCGACC | | |
| | A 3′ | C 3′ | | |
| Tmeff1 | 5′ gcGAGGCAGA | 5′ gcgCCGTTAT | 53 | 277 |
| | GGCAAGAGCATCA | CAGAGTAGCAAGG | | |
| | 3′ | T 3′ | | |
| Tnfrsf12a | 5′ gcgCGGGTTG | 5′ gcgAACCAGG | 59 | 200 |
| | GTGTTGATACGC | GCCAGACTAAGAG | | |
| | 3′ | C 3′ | | |
| Cyclin A2 | 5′ gcgTATTTGC | 5′ gcgCTGTGGT | 53 | 162 |
| | CATCGCTTATTGC | GCTTTGAGGTAGG | | |
| | T 3′ | T 3′ | | |
| PEA3 | 5′ gcgTCCCTGC | 5′ gcGATTTCTC | 57 | 782 |
| | CGCCTTCCGATTC | ATAGCCATAACCC | | |
| | A 3′ | 3′ | | |
| FHL1 | 5′ gcGTATTACT | 5′ gcgATTATTT | 53 | 440 |
| | GCGTGGATTGCTA | TTGCTGCGAGGTT | | |
| | 3′ | G 3′ | | |
| CSRP1 | 5′ gcGAGAGGTG | 5′ gcGATGGGCA | 54 | 415 |
| | CGGATAGGATTGT | AGGGAGCGAAGGT | | |
| | 3′ | 3′ | | |
| SHARP1 | 5′ gcgTCGGCTC | 5′ gcGAACTTGG | 53 | 487 |
| | TCTCGTGGCGTTG | AAACCTGGCGACT | | |
| | G 3′ | 3′ | | |
| IRX4 | 5′ gcgCTACCCG | 5′ gcGCAGGACC | 58 | 741 |
| | CAGTTTGGATACC | TTCGCTCTTGACA | | |
| | C 3′ | 3′ | | |
| PEA15 | 5′ gcgTCGCTGG | 5′ gcgGCTGGGG | 59 | 449 |
| | CTCTCTGGACTTG | ATACGGGTTA 3′ | | |
| | A 3′ | | | |
| CABC1 | 5′ gcgATGCCCA | 5′ gcGCTCTGCC | 61 | 284 |
| | AAGCCTGCCGTCC | TCACCCGCTCAAA | | |
| | T 3′ | 3′ | | |
| DAPK1 | 5′ gcgTGAGCGT | 5′ gcGCGAAGTA | 54 | 217 |
| | GAGGAGCCGAAGC | GTCATAGCAACAG | | |
| | A 3′ | 3′ | | |
| GAPDH | 5′ ACTCACTCAA | 5′ GTCATGAGCC | 55 | 102 |
| | GATTGTCAGCAAT | CTTCCACAATGCC | | |
| | G 3′ | A 3′ | | |
| β-actin | 5′ GGAGAAGATT | 5′ CAGGGAGGAA | 55 | 462 |
| | TGGCACCACAC | GAGGATGCGGC | | |
| | 3′ | 3′ | | |

*gcg clamps were added to primers to increase PCR efficiency.

1. Use of a compound comprising a p38 inhibitor or a pharmaceutically acceptable derivative thereof in the manufacture of a medicament for treatment of a condition or disease state to stimulate de-differentiation of post-mitotic cells.

2. The method of claim 1, wherein the post-mitotic cells are cardiomyocytes.

3. The method of claim 1 wherein the compound is selected from one or more of the classes of p38 inhibitors (A)-(I) described in the specification and pharmaceutically acceptable derivatives thereof.

4. The method of claim 1 wherein the compound is selected from 6-{5-[(cyclopropylamino)carbonyl]-3-fluoro-2-methylphenyl}-N-(2,2-dimethylpropyl)-3-pyridinecarboxamide 1-oxide; 6-{5-[(cyclopropylamino)carbonyl]-3-fluoro-2-methylphenyl}-N-[(1R)-1,2,2-trimethylpropyl)]-3-pyridinecarboxamide 1-oxide; 6-{5-[(cyclopropylamino)carbonyl]-3-fluoro-2-methylphenyl}-N-(1,1-dimethylpropyl)-3-pyridinecarboxamide 1-oxide; 6-{5-[(cyclopropylamino)carbonyl]-3-fluoro-2-methylphenyl}-N-(1-ethylpropyl)-3-pyridinecarboxamide 1-oxide; 6-{5-[(cyclopropylamino)carbonyl]-3-fluoro-2-methylphenyl}-N-[(1S)-1,2,2-trimethylpropyl)]-3-pyridinecarboxamide 1-oxide; 6-{5-[(cyclopropylamino)carbonyl]-3-fluoro-2-methylphenyl}-N-[(1R)-1,2-trimethylpropyl)]-3-pyridinecarboxamide 1-oxide; 6-{5-[(cyclopropylamino)carbonyl]-3-fluoro-2-methylphenyl}-N-[(1S)-1,2-trimethylpropyl)]-3-pyridinecarboxamide 1-oxide; 6-{5-[(cyclopropylamino)carbonyl]-3-fluoro-2-methylphenyl}-N-[(3,4-dimethylphenyl)methyl]-3-pyridinecarboxamide 1-oxide, and pharmaceutically acceptable derivatives thereof.

5. A method of inducing division of post mitotic cells, the method comprising
administering a p38 inhibitor or a pharmaceutically acceptable derivative thereof to a subject in an amount effective to stimulate de-differentiation of post-mitotic cells.

6. The method of claim 5, wherein the post-mitotic cells are cardiomyocytes.

7. The method of claim 5 wherein the p38 inhibitor or derivative thereof further comprises a compound selected from the group of formula (A)-(I) described in the specification, and pharmaceutically acceptable derivatives thereof.

8. The method of claim 5 wherein the p38 inhibitor or derivative thereof further comprises a compound selected from the group of 6-{5-[(cyclopropylamino)carbonyl]-3-fluoro-2-methylphenyl}-N-(2,2-dimethylpropyl)-3-pyridinecarboxamide 1-oxide; 6-{5-[(cyclopropylamino)carbonyl]-3-fluoro-2-methylphenyl}-N-[(1R)-1,2,2-trimethylpropyl)]-3-pyridinecarboxamide 1-oxide; 6-{5-[(cyclopropylamino)carbonyl]-3-fluoro-2-methylphenyl}-N-(1,1-dimethylpropyl)-3-pyridinecarboxamide 1-oxide; 6-{5-[(cyclopropylamino)carbonyl]-3-fluoro-2-methylphenyl}-N-(1-ethylpropyl)-3-pyridinecarboxamide 1-oxide; 6-{5-[(cyclopropylamino)carbonyl]-3-fluoro-2-methylphenyl}-N-[(1S)-1,2,2-trimethylpropyl)]-3-pyridinecarboxamide 1-oxide; 6-{5-[(cyclopropylamino)carbonyl]-3-fluoro-2-methylphenyl}-N-[(1R)-1,2-trimethylpropyl)]-3-pyridinecarboxamide 1-oxide; 6-{5-[(cyclopropylamino)carbonyl]-3-fluoro-2-methylphenyl}-N-[(1S)-1,2-trimethylpropyl)]-3-pyridinecarboxamide 1-oxide; 6-{5-[(cyclopropylamino)carbonyl]-3-fluoro-2-methylphenyl}-N-[(3,4-dimethylphenyl)methyl]-3-pyridinecarboxamide 1-oxide, and pharmaceutically acceptable derivatives thereof.

9. The method of claim 5, wherein the step of administering an effective amount of p38 inhibitors is selected from the group comprising oral administration, intravenous injection, topical administration, and myocardial injection.

10. The method of claim 5, wherein the step of administration comprises implanting a stent in the subject, such that the stent is capable of delivering p38 inhibitors to the subject's organ.

11. The method of claim 5, where the method upregulates cyclin A2.

12. A method of repairing heart tissue, the method comprising
identifying a subject in need of heart tissue repair, and
administering to the subject an effective amount of p38 inhibitor, such that proliferation of cardiomyocytes increases.

13. The method of claim 12, wherein the subject underwent myocardial ischemia, hypoxia, stroke, or myocardial infarction.

14. The method of claim 13, wherein the method further comprises administering an effective amount of FGF1, wherein the p38 inhibitor and FGF1 act synergistically to induce proliferation of cardiomyocytes.

15. The method of claim 13, wherein the method downregulates antagonists of PI3 kinase.

16. The method of claim 13, wherein the antagonist of PI3 kinase is Seta/Ruk.

17. A method for producing de-differentiated of cardiomyocytes comprising the steps of:
selecting terminally differentiated cells from a tissue that includes said cells;
resuspending said concentrated cells in a growth medium containing an effective amount of p38 inhibitor; and
culturing said resuspended cells in the growth medium for a time and under conditions to effect de-differentiation of at least a portion of said selected cells in culture,
wherein at least a portion of said selected terminally differentiated cells in culture undergo at least one round of cardiomyocyte division.

18. The method of claim 17, wherein the growth medium comprises FGF1.
